(19) United States
(12) Patent Application Publication
(10) Pub. No.: US 2024/0327873 A1
(43) Pub. Date: Oct. 3, 2024

(54) PROCESS REDUCING ENERGY CONSUMPTION IN GAS FERMENTATION

(71) Applicant: LanzaTech, Inc., Skokie, IL (US)

(72) Inventors: Steven Andrew Metzgar, Evanston, IL (US); Robert John Conrado, Washington, DC (US); Rachel Jane Brenc, Elmhurst, IL (US)

(21) Appl. No.: 18/617,316

(22) Filed: Mar. 26, 2024

Related U.S. Application Data
(60) Provisional application No. 63/493,933, filed on Apr. 3, 2023.

(51) Int. Cl. C12P 1/04; B01D 53/04; B01D 53/047; B01D 53/14; B01D 53/22; C01B 32/50

(52) U.S. Cl. CPC C12P 1/04; B01D 53/0462; B01D 53/047; B01D 53/1425; B01D 53/226; C01B 32/50; B01D 2252/204; B01D 2256/22; B01D 2259/40013; B01D 2259/40028; B01D 2259/40062; B01D 2259/40081; B01D 2259/403; B01D 2259/404; B01D 2259/4062; C01B 2210/0007; C01B 2210/001; C01B 2210/0014

(57) ABSTRACT

Converting carbon sources that would otherwise be vented to the atmosphere or discarded as waste to one or more products. Carbon sources may be dilute carbon containing streams that are purified to from about 90 vol.-% to about 95 vol.-% carbon compound. In certain aspects, also disclosed are the processes for producing desirable products, such as ethylene, from industrial waste streams.

### CROSS REFERENCE TO RELATED APPLICATIONS

This application claims the benefit of U.S. Provisional Patent Application No. 63/493,933 filed on Apr. 3, 2023, the entirety of which is incorporated herein by reference.

### FIELD

The present disclosure relates to improving carbon transformation and utilization and. reducing energy consumption in gas fermentation processes by modifying low energy carbon dioxide purification techniques to produce a gas fermentation feedstock stream comprising from about 80 vol.-% to about 95 vol.-% carbon dioxide or from about 90 vol.-% to about 95 vol.-% carbon dioxide while at the same time reducing carbon dioxide emissions into the atmosphere from low concentration carbon dioxide waste or underutilized streams that may be otherwise too costly due to high energy consumption to concentrate for conversion by microbial gas fermentation. Such carbon dioxide sources would otherwise be vented to the atmosphere or discarded to one or more products. More specifically, the present disclosure relates to systems and methods for purifying carbon dioxide-containing streams for microbial gas fermentation to convert underutilized resources into useful products, such as ethylene, ethanol, and the like.

### BACKGROUND

The following discussion is provided to aid the reader in understanding the disclosure and is not admitted to describe or constitute prior art thereto.

Mitigation of impending climate change requires drastic changes in manufacturing and greater reliance on biotechnology. Sustainable sources of fuels and chemicals are currently insufficient to significantly displace dependence on fossil carbon. Biotechnology harnesses the power of biology to create new products in a way that improves the quality of life and the environment, Gas fermentation is emerging as a powerful biotechnological advancement as an alternative platform for the biological fixation of such gases such as CH₄, CO, CO₂, and/or H₂ into sustainable fuels and chemicals. Gas fermentation processes can be used to generate target materials from gas substrates or other input materials, particularly carbon-based materials. For example, particular biological systems can be used to perform gas fermentation.

Although industrial processes can output gases that have significant amounts of carbon-based materials such as carbon dioxide, many output gases may to too dilute for use directly in gas fermentation and too expensive to purify using existing techniques. Thus flaring and venting carbon sources to the atmosphere or otherwise discarding them can be viewed as traditional standard operations. A primary alternative available to industry is to engage in some form of carbon capture and sequestration (“CCS”).

CCS can include finding permanent underground storage such as depleted oil wells or sealed saline aquifers to permanently store the gaseous carbon. However, CCS requires the carbon sources to be purified to 99+ vol.-% purity before being considered suitable for CCS. This may be cost prohibitive for operators that produce waste or underutilized carbon, as it requires them to construct expensive and energy intensive processes to purify the carbon source to the very high degree of purity required for CCS.

Additionally, many domestic and international governmental entities are placing tighter restrictions on the total amount of carbon that a particular site, complex, or entity is allowed to release into the atmosphere. Such restrictions are pushing industrial, commercial, and agricultural operators alike to pursue and implement expensive efficiency upgrades to already well-developed technologies within their respective fields.

Although gas fermentation processes can be used for carbon capture and other applications, gas fermentation could be more widely implemented to address a larger scope of waste or underutilized carbon containing sources if cost barriers of purifying the waste or underutilized carbon source for gas fermentation processing could be reduced, such as by employing low energy intensive techniques configured and operated to provide carbon feedstock at a concentration uniquely appropriate for microbial gas fermentation. As with any disruptive technology, many technical challenges must be overcome before this potential is fully achieved. The science of scale-up production and the reduction of obstacles for continued commercialization of gas fermentation are advanced by this disclosure.

### SUMMARY

In a first aspect, the present disclosure provides methods and systems of carbon utilization comprising: a) passing a dilute carbon dioxide stream to a purification zone wherein the dilute carbon dioxide stream comprises less than about 80 vol.-% carbon dioxide; b) purifying the dilute carbon dioxide stream to generate a purified carbon dioxide stream comprising from about 80 vol.-% to about 95 vol.-% carbon dioxide, wherein the purifying is by a process selected from an amine absorption process, a temperature swing adsorption process, a pressure swing adsorption process, a membrane separation process, or any combination thereof; c) passing hydrogen and the purified carbon dioxide stream to a gas fermentation zone and contacting with C1-fixing microorganism biocatalyst to ferment at least the hydrogen and the purified carbon dioxide stream and produce at least one target product; and d) recovering the target product.

In some embodiments, the dilute carbon dioxide stream comprises less than about 90 vol.-% carbon dioxide, and the purified carbon dioxide stream comprises from about 90 vol.-% to about 95 vol.-% carbon dioxide.

In some embodiments, the C1-fixing microorganism is an acetogenic carboxydotrophic microorganism.

In some embodiments the purification process is amine absorption, and the solvent of the amine absorption process is selected from aqueous solutions of monoethanolamine, piperazine, diethanolamine, methyldiethanolamine, diglycolamine, 2-amino-2-methyl-1-propanol, or any combination thereof.

In some embodiments the amine absorption purification process comprises an absorber column and a desorber column, wherein a rich solvent from the absorber column is preheated by heat exchange with regenerated solvent from the desorber column.

In some embodiments the amine absorption purification process comprises an absorber column and a desorber column, and a rich solvent from the absorber column is split into two or more rich solvent streams and each rich solvent stream is passed to the desorber column.

In some embodiments the purification process is pressure swing adsorption comprising at least four adsorbent beds wherein the pressure swing adsorption cycle comprises: a) passing the dilute carbon dioxide stream through the first bed at a first pressure to generate a light product stream; b) depressurizing the second bed in a counter current mode from the first pressure to a second pressure lower than the first pressure generating a heavy product stream comprising carbon dioxide; c) desorbing the third bed at the second pressure using a first portion of the light product stream to generate a second heavy product stream comprising carbon dioxide; d) repressurizing the fourth bed from the second pressure to a third pressure higher than the second pressure using a second portion of the light product stream; and e) repeating the cycle to generate the purified carbon dioxide stream comprising from about 80 vol.-% to about 95 vol.-% carbon dioxide.

In some embodiments the light product stream comprises nitrogen.

In some embodiments the third pressure is about the same as the first pressure.

In some embodiments the purification process is pressure swing adsorption comprising at least four adsorbent beds wherein the pressure swing adsorption cycle comprises: a) passing the dilute carbon dioxide stream through the first bed at a first pressure to generate a first light product stream; b) passing a portion of a heavy product stream from the third bed through the second bed at the first pressure in a counter current mode generating a second light product stream; c) depressurizing the third bed in a counter current mode from the first pressure to a second pressure lower than the first pressure generating the heavy product stream comprising carbon dioxide; d) repressurizing the fourth bed from the second pressure to a third pressure higher than the second pressure using a portion of the first light product stream, the second light product stream, or a combination thereof; and e) repeating the cycle to generate the purified carbon dioxide stream comprising from about 80 vol.-% to about 95 vol.-% carbon dioxide.

In some embodiments the light product stream comprises nitrogen.

In some embodiments the third pressure is about the same as the first pressure.

In some embodiments the purification process is pressure swing adsorption comprising at least five adsorbent beds wherein the pressure swing adsorption cycle comprises: a) passing the dilute carbon dioxide stream through the first bed at a first pressure to generate a first light product stream; b) passing at least a portion of a first or a second heavy product stream through the second bed at the first pressure in a counter current mode generating a second light product stream; c) depressurizing the third bed in a counter current mode from the first pressure to a second pressure lower than the first pressure generating the first heavy product stream comprising carbon dioxide; d) desorbing the fourth bed at the second pressure using a first portion of the light product stream to generate a second heavy product stream comprising carbon dioxide; c) repressurizing the fifth bed from the second pressure to a third pressure higher than the second pressure using a second portion of the first light product stream, a portion of the second light product stream, or a combination thereof; and f) repeating the cycle to generate the purified carbon dioxide stream comprising from about 80 vol.-% to about 95 vol.-% carbon dioxide.

In some embodiments the light product stream comprises nitrogen.

In some embodiments the third pressure is about the same as the first pressure.

In some embodiments the purification process is temperature swing adsorption comprising at least three adsorbent beds wherein the temperature swing adsorption cycle comprises: a) passing the dilute carbon dioxide stream through the first bed at a first temperature to generate a light product stream; b) heating the second bed in a counter current mode from the first temperature to a second temperature higher than the first temperature generating a heavy product stream comprising carbon dioxide; c) cooling the third bed from the second temperature to a third temperature lower than the second temperature; and d) repeating the cycle to generate the purified carbon dioxide stream comprising from about 80 vol.-% to about 95 vol.-% carbon dioxide.

In some embodiments the light product stream comprises nitrogen.

In some embodiments the third temperature is about the same as the first temperature.

In some embodiments the purification process is temperature swing adsorption comprising at least five adsorbent beds wherein the temperature swing adsorption cycle comprises: a) passing the dilute carbon dioxide stream through the first bed at a first temperature to generate a light product stream; b) passing an effluent from the fourth bed through the second bed at a second temperature higher than the first temperature generating a second light product stream; c) heating the third bed in a counter current mode from the second temperature to a third temperature higher than the second temperature generating a heavy product stream comprising carbon dioxide; d) passing at least a portion of the first or a portion of the second light product stream through the fourth bed at the second temperature in a counter current mode generating the effluent stream from the fourth bed; c) cooling the fifth bed from the second temperature to a fourth temperature lower than the second temperature; and f) repeating the cycle to generate the purified carbon dioxide stream comprising from about 80 vol.-% to about 95 vol.-% carbon dioxide.

In some embodiments the light product stream comprises nitrogen.

In some embodiments the fourth temperature is about the same as the first temperature.

In some embodiments the purification process is temperature swing adsorption comprising at least six adsorbent beds wherein the temperature swing adsorption cycle comprises: a) mixing the dilute carbon dioxide stream and an effluent from the third bed at a second temperature to preheat the dilute carbon dioxide stream to a first temperature and passing the mixture through the first bed at the first temperature to generate a first light product stream; b) passing an effluent from the fifth bed through the second bed in a counter current mode at the first temperature to generate a second light product stream; c) heating the third bed in a counter current mode from the first temperature to the second temperature higher than the first temperature generating the effluent from the third bed at the second temperature; d) desorbing the fourth bed at the second temperature to generate a heavy product stream comprising carbon dioxide; c) passing at least a portion of the first light product stream, a portion of the second light product stream, or both through the fifth bed at the first temperature in a co current mode generating the effluent stream from the fifth bed; f) cooling the sixth bed the second temperature to a third temperature lower than the second temperature; and g) repeating the cycle to generate the purified carbon dioxide stream comprising from about 80 vol.-% to about 95 vol.-% carbon dioxide.

In some embodiments the light product stream comprises nitrogen.

In some embodiments the purification process is a multi-stage membrane process comprising at least n membrane stages wherein a retentate of each membrane stage is passed to the feed of membrane stage n+1, and the permeate of each membrane stage is recycled to membrane stage n−1.

In some embodiments n=2 or n=3.

In some embodiments, the purification process is a membrane suitable to generate the purified carbon dioxide stream.

In some embodiments the dilute carbon dioxide stream is provided from an industrial process.

In some embodiments the dilute carbon dioxide stream is a waste gas, flare gas, or flue gas.

In some embodiments the microorganism is selected from a genus of Clostridium, Moorella, Carboxydothermus, Ruminococcus, Acetobacterium, Eubacterium, Butyribacterium, Oxobacter, Methanosarcina, Methanosarcina, Desulfotomaculum, and Cupriavidus.

In some embodiments the target product is selected from an alcohol, an acid, a diacid, an alkene, a terpene, an isoprene, and alkyne.

### BRIEF DESCRIPTION OF THE DRAWINGS

The figures have been simplified by the deletion of a large number of apparatuses customarily employed in a process/system of this nature, such as vessel internals, temperature and pressure control systems, flow control valves, recycle pumps and the like, which are not specifically required to illustrate the performance of the disclosure. Furthermore, the illustration of the process of this disclosure in the embodiment of a specific drawing is not intended to limit the disclosure to specific embodiments. Some embodiments may be described by reference to the process configuration shown in the figures, which relates to both apparatus and processes to carry out the disclosure. Any reference to a process step includes reference to an apparatus unit or equipment that is suitable to carry out the step, and vice-versa.

FIG. 1 is an overview of the piping and associated components of an embodiment of a gas fermentation process.

FIG. 2 is an overview of the piping and associated components of an embodiment of a gas fermentation process wherein the purification process of a dilute carbon dioxide stream is an amine absorption process.

FIGS. 3a, 3b, and 3c are overviews of the piping and associated components of embodiments of a gas fermentation process wherein the purification process of a dilute carbon dioxide stream are pressure swing adsorption processes.

FIGS. 4a, 4b, and 4c are overviews of the piping and associated components of embodiments of a gas fermentation process wherein the purification process of a dilute carbon dioxide stream are temperature swing adsorption processes.

FIG. 5 is an overview of the piping and associated components of an embodiment of a gas fermentation process wherein the purification process of a dilute carbon dioxide stream is a multistage membrane separation process.

### DETAILED DESCRIPTION

The present disclosure provides systems and methods for improving carbon capture and utilization by unlocking a wider variety of carbon sources suitable for microbial gas fermentation that converts carbon sources that would otherwise be vented to the atmosphere or discarded to one or more products. More specifically, the present disclosure relates to systems and methods for purifying dilute carbon containing streams using configurations and conditions requiring less energy than typical carbon dioxide purification techniques employed today to generate a stream suitable for microbial gas fermentation. Therefore, a greater amount of carbon such as carbon dioxide may be converted into useful products, such as ethylene, ethanol, and the like instead of being vented to the atmosphere.

### A. Definitions

It is to be understood that the terminology used herein is for the purpose of describing particular embodiments only and is not intended to be limiting.

Technical and scientific terms used herein have the meanings commonly understood by one of ordinary skill in the art, unless otherwise defined. Unless otherwise specified, materials and/or methodologies known to those of ordinary skill in the art can be utilized in carrying out the methods described herein, based on the guidance provided herein.

As used herein, the singular terms “a,” “an,” and “the” include plural referents unless the context clearly dictates otherwise. Reference to an object in the singular is not intended to mean “one and only one” unless explicitly so stated, but rather “one or more.”

As used herein, “about” when used with a numerical value means the numerical value stated as well as plus or minus 10% of the numerical value. For example, “about 10” should be understood as both “10” and “9-11.”

Also as used herein, “and/or” refers to and encompasses any and all possible combinations of one or more of the associated listed items, as well as the lack of combinations when interpreted in the alternative (“or”).

The term “acid” as used herein includes both carboxylic acids and the associated carboxylate anion, such as the mixture of free acetic acid and acetate present in a fermentation broth as described herein. The ratio of molecular acid to carboxylate in the fermentation broth is dependent upon the pH of the system. In addition, the term “acetate” includes both acetate salt alone and a mixture of molecular or free acetic acid and acetate salt, such as the mixture of acetate salt and free acetic acid present in a fermentation broth as described herein.

The term “carbon capture” as used herein refers to the fixation and utilization of carbon including carbon from CO₂, CO, and/or CH₄ from a stream comprising CO₂, CO, and/or CH₄ and converting the CO₂, CO, and/or CH₄ into useful products.

The term “substrate comprising carbon monoxide” and like terms should be understood to include any substrate in which carbon monoxide is available to one or more strains of bacteria for growth and/or fermentation, for example.

The term “gaseous substrates comprising carbon monoxide” includes any gas which contains carbon monoxide. The gaseous substrate will typically contain a significant proportion of CO, preferably at least about 5 vol.-% to about 100 vol.-% CO.

The term “C1 carbon” and like terms should be understood to refer to carbon sources that are suitable for use by a C1 fixing microorganism, particularly those of the gas fermentation process disclosed herein. C1 carbon may include, but should not be limited to, carbon monoxide (CO), carbon dioxide (CO₂), and methane (CH₄), methanol (CH₃OH), and formate (HCOOH).

The term “substrate comprising carbon dioxide” and like terms should be understood to include any substrate in which carbon dioxide is available to one or more strains of bacteria for growth and/or fermentation, for example.

The term “gaseous substrates comprising carbon dioxide” includes any gas which contains carbon dioxide. Some gaseous substrates contain a significant proportion of CO₂, such as at least about 50 vol.-% to about 100 vol.-% CO₂, while other gaseous substrates that contain CO₂ are more dilute and contain from about 5 vol.-% to about 50 vol.-% Advantageously the present disclosure reduces energy requirement for gaseous substrates comprising from about 5 vol.-% to about 20 vol.-% carbon dioxide thereby allowing dilute CO₂ containing streams to be processed economically by gas fermentation.

The term “bioreactor” includes a fermentation device consisting of one or more vessels and/or towers or piping arrangements, which includes the continuous stirred tank reactor (CSTR), immobilized cell reactor (ICR), trickle bed reactor (TBR), bubble column, gas lift fermenter, loop reactors, membrane reactor such as hollow fiber membrane bioreactor (HFMBR), static mixer, high throughput, or other vessel or other device suitable for gas-liquid contact.

The term “co-substrate” refers to a substance that, while not necessarily being the primary energy and material source for product synthesis, can be utilized for product synthesis when added to another substrate, such as the primary substrate.

The term “directly”, as used in relation to the passing of industrial off or gases to a bioreactor, is used to mean that no or minimal processing or treatment steps, such as cooling and particulate removal are performed on the gases prior to them entering the bioreactor (note: an oxygen removal step may be required for anaerobic fermentation).

The terms “fermenting,” “fermentation process,” “fermentation reaction,” and like terms as used herein, are intended to encompass both the growth phase and product biosynthesis phase of the process. As is described further herein, in some embodiments the bioreactor may comprise a primary bioreactor and a secondary bioreactor.

The term “nutrient medium” as used herein should be understood as the solution added to the fermentation broth containing nutrients and other components appropriate for the growth of the microorganism culture.

The terms “primary bioreactor” or “first reactor” as used herein this term is intended to encompass one or more reactors that may be connected in series or parallel with a secondary bioreactor. The primary bioreactors generally use anaerobic or aerobic fermentation to produce a product (e.g., ethylene, ethanol, acetate, etc.) from a gaseous substrate.

The terms “secondary bioreactor” or “second reactor” as used herein are intended to encompass any number of further bioreactors that may be connected in series or in parallel with the primary bioreactors. Any one or more of these further bioreactors may also be connected to a further separator.

The term “stream” is used to refer to a flow of material into, through and away from one or more stages of a process, for example, the material that is fed to a bioreactor. The composition of the stream may vary as it passes through particular stages. For example, as a stream passes through the bioreactor.

The terms “feedstock” when used in the context of the stream flowing into a gas fermentation bioreactor (i.e., gas fermenter) or “gas fermentation feedstock” should be understood to encompass any material (solid, liquid, or gas) or stream that can provide a substrate and/or C1-carbon source to a gas fermenter or bioreactor either directly or after processing of the feedstock.

The term “waste gas” or “waste gas stream” may be used to refer to any gas stream that is either emitted directly, flared with no additional value capture, or combusted for energy recovery purposes.

The term “underutilized gas” or “underutilized gas stream” may be used to refer to any gas stream that may have greater value as a substrate to gas fermentation than a current use.

The terms “synthesis gas” or “syngas” refers to a gaseous mixture that contains at least one carbon source, such as carbon monoxide (CO), carbon dioxide (CO₂), or any combination thereof, and, optionally, hydrogen (H₂) that can used as a feedstock for the disclosed gas fermentation processes and can be produced from a wide range of carbonaceous material, both solid and liquid.

### B. Disclosed Systems and Methods

For many industrial processes, emission of gases that contain carbon are commonplace. Industrial process operators may view flaring and venting carbon rich sources to the atmosphere or otherwise discarding them as traditional standard techniques. Many domestic and international governmental entities are placing tighter restrictions on the total amount of waste carbon that a particular site, complex, or entity is allowed to release into the atmosphere. Such restrictions are pushing industrial, commercial, and agricultural operators alike to pursue and implement expensive efficiency upgrades to perhaps already well-developed technologies within their respective fields.

Currently, the primary alternative available to industrial operators is to engage in some form of carbon capture and sequestration (“CCS”). CCS involves finding permanent underground storage such as empty oil wells, gas-tight saline aquifers, or salt domes to permanently store the waste gaseous carbon. This option is costly for operators that produce waste carbon, as it requires them to locate a suitable location, construct a pipeline to that location, and then monitor the location indefinitely for leaks or other signs of failure. Furthermore, CCS typically requires the carbon gases to be of very high purity such as greater than 99 vol.-% by volume of the carbon compound. For example, a CO₂ stream would need to be at least 99 vol.-% CO₂.

With the primary alternative to flaring being CCS, and CCS requiring purity of at least 99 vol.-% by volume of carbon compounds, current purification technology and processes are directed to achieving at least 99 vol.-% by volume of carbon compounds. After reaching a certain purity level, the energy requirements to progress to at least 99 vol.-% by volume purity increase exponentially. Thus, significant energy costs are required to reach at least 99-vol. % by volume purity required for CCS.

Systems and methods in accordance with the present disclosure can be used to transform carbon in dilute carbon containing gas streams by microbial gas fermentation systems to generate valuable products and divert carbon compounds from being emitted into the atmosphere without incurring the high energy costs required for CCS of the same stream.

Configuring and operating purification processes tailored to the unique gas fermentation (hereinafter referred to as “GF”) unit requirements allow operators with carbon containing waste or underutilized streams having less than 80 vol.-% or less than 90 vol.-% carbon compound to reduce their overall carbon emissions by converting these underutilized and/or waste carbon into marketable products without the high level energy costs for purification as required by CCS.

Gas fermentation processes that are capable of converting various carbon sources into other products are rapidly becoming a desirable alternative for producers with excess carbon. Such processes allow companies or organizations to convert standard techniques that emit carbon into the atmosphere into a separate revenue stream by converting the waste or underutilized carbon into a marketable product. Moreover, the carbon that is converted into other products lowers the operator's total carbon output, potentially serving as a way for operators to maintain current outputs without conflicting with ever-tightening government regulations. Furthermore, tail gas from gas fermentation may be another source of CO₂ and purified to form a concentrated CO₂ stream thereby further reducing cost as compared to more traditional carbon capture and sequestration processes.

The widespread adoption of gas fermentation processes could be improved by reducing the cost barriers of employing dilute carbon streams, especially dilute CO₂ streams, as carbon sources for gas fermentation. Many industrial processes generate dilute CO₂ steams that are too energy cost prohibitive using current purification techniques to be used as carbon sources for gas fermentation. Current purification equipment and techniques are designed to purify CO₂ streams for CCS, which typically requires greater than 99 vol.-% CO₂. Because of the availability of dilute CO₂ stream in particular, the disclosure will be discussed in terms of CO₂ as the carbon source for a GF system. However, such exemplary discussion is not intended to limit the full scope to CO₂ streams, and other carbon sources may be employed instead of or in addition to the exemplary CO₂ streams. Modifying and operating CO₂ purification techniques typically used for CCS in a manner specifically tailored to the unique requirements of gas fermentation systems and processes significantly reduces energy requirements and unlocks dilute CO₂ streams as realistic carbon sources for microbial gas fermentation.

In one embodiment the substrate and/or C1 carbon source provides both the energy and the carbon source for the metabolic process of the biocatalyst, while in another embodiment, such as when CO₂ is the carbon source, depending upon the biocatalyst, a source of energy for the metabolic process is also provided. Typically, the source of energy for the metabolic process may be hydrogen. The hydrogen may be mixed with the C1 carbon source prior to the bioreactor of the gas fermentation system, or may be independently supplied to the bioreactor.

The substrate and or C1 carbon source may be already in the form of a gas (e.g., a waste gas), or a solid or liquid material may be first processed in a preliminary step of the overall gas fermentation process to generate synthesis gas known as syngas which in turn is provided to the bioreactor of the gas fermentation system. The preliminary step to generate syngas may involve reforming, partial oxidation, plasma, or gasification processes. Examples of gasification processes include gasification of coal, gasification of refinery residues, gasification of petroleum coke, gasification of biomass, gasification of lignocellulosic material, gasification of waste wood, gasification of black liquor, gasification of municipal solid waste, gasification of municipal liquid waste, gasification of industrial solid waste, gasification of industrial liquid waste, gasification of refuse derived fuel, gasification of sewerage, gasification of sewerage sludge, gasification of sludge from wastewater treatment, gasification of landfill gas, gasification of biogas such as when biogas is added to enhance gasification of another material. Examples of reforming processes include, steam methane reforming, steam naphtha reforming, reforming of natural gas, reforming of biogas, reforming of landfill gas, reforming of coke oven gas, reforming of pyrolysis off-gas, reforming of ethylene production off-gas, naphtha reforming, and dry methane reforming. Examples of partial oxidation processes include thermal and catalytic partial oxidation processes, catalytic partial oxidation of natural gas, partial oxidation of hydrocarbons, partial oxidation of biogas, partial oxidation of landfill gas, or partial oxidation of pyrolysis off-gas. Examples of municipal solid waste include tires, plastics, refuse derived fuel, and fibers such as in shoes, apparel, and textiles. Municipal solid waste may be simply landfill-type waste and may be sorted or unsorted. Examples of biomass may include lignocellulosic material and microbial biomass. Lignocellulosic material may include agriculture waste and forest waste.

The substrate and/or C1-carbon source may be a gas stream comprising methane. Such a methane containing gas may be obtained from fossil methane emissions such as during fracking or other hydrocarbon well stimulation processes or from coalbeds, or may be obtained from wastewater treatment, livestock, agriculture, and municipal solid waste landfills. It is also envisioned that the methane may be burned or employed as a feed in a fuel cell to produce electricity or heat, and the C1 by-products may be used as the substrate or carbon source.

The microorganism of the disclosure may be cultured with the gaseous substrate to produce one or more target products. Target product(s) may be selected from an alcohol, an acid, a diacid, an alkene, a terpene, an isoprene, and alkyne. For instance, the microorganism of the disclosure may produce or may be engineered to produce ethanol, acetate, 1-butanol, 2,3-butanediol, lactate, butene, butadiene, methyl ethyl ketone (2-butanone), ethylene, acetone, isopropanol, lipids, 3-hydroxypropionate (3-HP), terpenes, including isoprene, fatty acids, 2-butanol, 1,2-propanediol, 1 propanol, 1 hexanol, 1 octanol, chorismate-derived products, 3 hydroxybutyrate, 1,3 butanediol, 2-hydroxyisobutyrate or 2-hydroxyisobutyric acid, isobutylene, adipic acid, 1,3 hexanediol, 3-methyl-2-butanol, 2-buten-1-ol, isovalerate, isoamyl alcohol, and/or monoethylene glycol in addition to ethylene. In certain embodiments, microbial biomass itself may be considered a product.

In certain embodiments, industrial process have material such as waste gas or underutilized gas that may be suitable as substrate and/or C1 carbon source for GF and are selected from ferrous metal products manufacturing, such as a steel manufacturing, non-ferrous products manufacturing, petroleum production, petroleum refining, electric power production, carbon black production, paper and pulp manufacturing, ammonia production, methanol production, coke manufacturing, coke gasification, petrochemical production, polymer production, ethylene production, olefin production, carbohydrate fermentation, cement making, aerobic digestion, anaerobic digestion, catalytic processes, natural gas extraction, cellulosic fermentation, oil extraction, industrial processing of geological reservoirs, processing fossil resources such as natural gas coal and oil, landfill operations, or any combination thereof.

Examples of specific processing steps within an industrial process which may generate substrate and/or C1 carbon source for gas fermentation include catalyst regeneration and fluid catalyst cracking. Air separation and direct air capture are other suitable industrial processes to provide substrate and/or C1 carbon source for the gas fermentation process. Specific examples in steel and ferroalloy manufacturing include blast furnace gas, basic oxygen furnace gas, coke oven gas, direct reduction of iron furnace top-gas, and residual gas from smelting iron. In petroleum and oil production, C1 carbon may be produced with the oil or may be produced from a well separately. Other general examples include flue gas from fired boilers and fired heaters, such as natural gas, oil, or coal fired boilers or heaters, flare gas, and gas turbine exhaust. In these embodiments, the substrate and/or C1-carbon source may be captured from the industrial process before it is emitted into the atmosphere, using any known method.

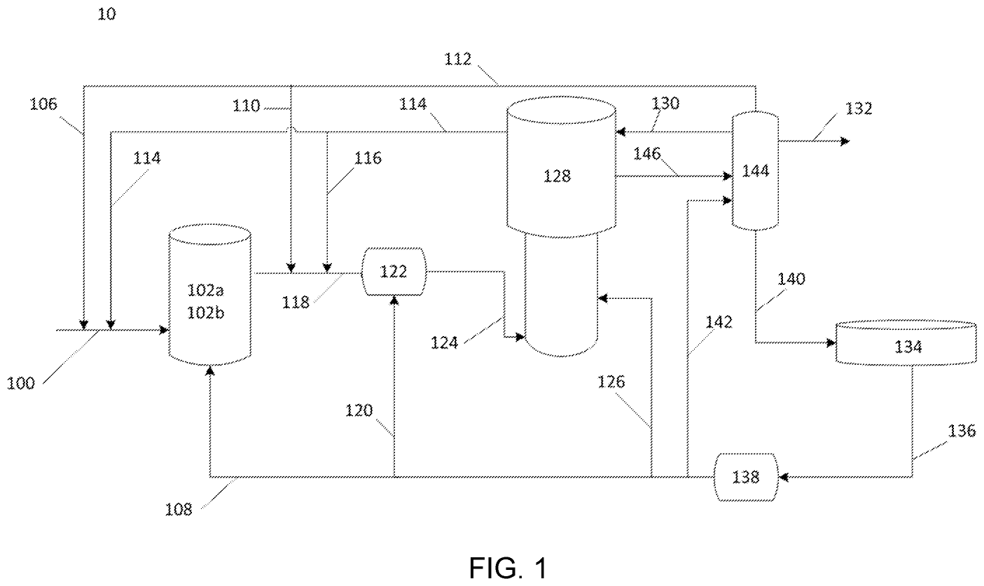

Referring now to FIG. 1, which illustrates a GF system 10 having a purification zone within the system 10, FIG. 1 shows an enlarged gas fermentation process including an optional gasification process 102a, a gas fermentation zone 128, a product recovery zone 144, and an optional wastewater treatment zone 134. Optional gasification process 102a receives feedstock 100, which may be any suitable material capable of being gasified to produce syngas stream 118. In various instances, feedstock 100 may be comprised at least partially of sorted and/or unsorted industrial or municipal solid waste including tires and end of life tires. In other instances, the feedstock 100 is comprised at least partially of forest and/or agricultural waste. In particular embodiments, feedstock 100 is comprised of any combination of two or more of the following: sorted municipal or industrial solid waste, unsorted municipal or industrial solid waste, forest waste, agricultural waste, or other solid or liquid waste from the refining or chemical process integrated with the enlarged gas fermentation process. Integration internal to the enlarged fermentation process would also provide for at least one effluent from the gas fermentation zone 128, at least one effluent from the product recovery zone 144, and or at least one effluent from the wastewater treatment zone 134 being used as gasification feed.

Gasification zone 102a is to produce syngas as substrate for gas fermentation zone 128. If a gas feedstock is already present for use as substrate for gas fermentation zone, gasification zone 102a may not be required. In some embodiments, syngas 118 produced in the gasification zone 102a by the gasification process, or gas obtained from another source contains one or more constituents that needs to be removed and/or converted. Typical constituents found in the syngas stream 118 that may need to be removed and/or converted include, but are not limited to, sulfur compounds, aromatic compounds, alkynes, alkenes, alkanes, olefins, nitrogen compounds, phosphorous-containing compounds, particulate matter, solids, oxygen, halogenated compounds, silicon-containing compounds, carbonyls, metals, alcohols, esters, ketones, peroxides, aldehydes, ethers, and tars. These constituents may be removed by one or more removal zones 122 positioned between gasification zone 102a and gas fermentation zone 128. Removal zone 122 may comprise one or more of the following modules: hydrolysis module, acid gas removal module, deoxygenation module, catalytic hydrogenation module, particulate removal module, chloride removal module, tar removal module, and hydrogen cyanide polishing module. Two or more modules may be combined into a single module performing the same functions. The functions of all modules may be combined into a single unit with the selection of an appropriate catalyst, such as for example U.S. Pat. No. 11,441,116. When incorporating removal process 122, at least a portion of syngas 118 from gasification zone 102a is passed to removal process 122 to remove and/or convert at least a portion of at least one constituent found in syngas stream 118. Removal zone 122 may operate to bring the constituent(s) within allowable levels to produce a treated stream 124 suitable for fermentation by in gas fermentation zone 128.

Fermentation process 128 employs at least one C1-fixing microorganism in a liquid nutrient media to ferment a feedstock gas, or syngas stream 124 and produce one or more products. The C1-fixing microorganism in fermentation process 128 may be a carboxydotrophic bacterium, or an acetogenic carboxydotrophic bacterium. In particular embodiments, the C1-fixing microorganism may be an acetogenic carboxydotrophic bacterium. The C1-fixing microorganism may be selected from the group comprising Moorella, Clostridium, Ruminococcus, Acetobacterium, Eubacterium, Butyribacterium, Oxobacter, Methanosarcina, Methanosarcina, Cupriavidus and Desulfotomaculum. In various embodiments, the acetogenic carboxydotrophic bacterium is Clostridium autoethanogenum.

The one or more products produced in fermentation zone 128 are removed and/or separated from the fermentation broth in product recovery zone 144. Product recovery zone 144 separates and removes one or more product(s) 132 and produces at least one effluent 142, 130, 112, which comprise reduced amounts of at least one product. Product depleted effluent may be sent via a conduit 142 to wastewater treatment zone 134 to produce at least one effluent 136, which may be recycled to the gasification process 102a and/or the fermentation process 128.

In at least one embodiment, an effluent from fermentation zone 128 is tail gas containing gas generated by the fermentation, inert gas, and or unmetabolized substrate. At least a portion of this tail gas may be passed via a conduit 114 to gasification zone 102a to be used as part of feedstock 100. At least a portion of the tail gas may be sent via conduit 114 and a conduit 116 to syngas 118, an effluent of gasification zone 102a, to quench syngas stream 118. At least a portion of the tail gas may be passed outside of the enlarged gas fermentation process.

In at least one embodiment, the effluent from fermentation zone 128 is fermentation broth. At least a portion of the fermentation broth may be sent via conduit 146 to product recovery zone 144. In at least one embodiment, product recovery zone 144 separates at least a portion of the microbial biomass from the fermentation broth. In various instances, at least a portion of the microbial biomass that is separated from the fermentation broth is recycled to the fermentation zone 128 via a conduit 130. In various instances, at least a portion of the microbial biomass separated from the fermentation broth is passed via a conduit 106 to optional gasification zone 102a for use as part of feedstock 100. In certain instances, fermentation zone 128 produces fusel oil which may also be recovered in product recovery zone 144 through any suitable means such as within the rectification column of a distillation system. In at least one embodiment, at least a portion of the fusel oil from the product recovery zone 144 is used as a heating source for one or more zones or elsewhere external to the enlarged process.

In various instances, at least a portion of a wastewater stream, comprising fermentation broth, which may contain microbial biomass from fermentation zone 128 may be passed via conduit 114 to optional gasification zone 102a, without being passed to product recovery zone 144.

In instances where the fermentation broth is processed by the product recovery process 144, at least a portion of the microbial biomass depleted water, produced through the removal of microbial biomass from the fermentation broth, may be returned to fermentation zone 128 via a conduit 130 and/or sent via a conduit 112 to optional gasification zone 102a. At least a portion of the microbial biomass depleted water may be passed via conduit 106 to optional gasification zone 102a to be used as part of feedstock 100. At least a portion of the microbial biomass depleted water may be passed via conduit 110 to quench syngas stream 118. At least a portion of the effluent from product recovery zone 144 may be passed via conduit 140 to wastewater treatment zone 134. The effluent from product recovery zone 144 may comprise reduced amounts of product and/or microbial biomass.

Wastewater treatment zone 134 receives and treats effluent from one or more zones to produce clarified water. The clarified water may be passed or recycled via a conduit 136 to one or more zones. For example, at least a portion of the clarified water may be passed via conduit 126 to the fermentation zone, at least a portion of the clarified water may be passed to optional gasification zone 102a via conduit 108 to be used as part of feedstock 100 and or via conduit 120 to quench syngas stream 118 in quench zone 122. In certain instances, the wastewater treatment process 134 generates microbial biomass as part of the treatment process. At least a portion of this microbial biomass may be passed via conduit 108 to the gasification zone 102a for use as part of feedstock 100. Wastewater treatment zone 134, as a by-product of treating microbial biomass, produces biogas. At least a portion of the biogas may be passed via conduit 136 to gasification zone 102a to be used as part of feedstock 100 and or via a conduit 120 to quench syngas stream 118.

Optional wastewater treatment effluent removal unit 138 is positioned downstream of wastewater treatment zone 134. At least a portion of biogas from wastewater treatment zone 134 may be passed to removal unit 138 to remove and/or convert at least a portion of at least one constituent found in the biogas stream. Removal unit 138 operates to lower the concentration of constituents to within predetermined allowable levels and produce a treated stream 142, 126, 120, and/or 108 suitable to be used by the subsequent one or more zones 144, 128, 122, and/or 102a, respectively.

Although FIG. 1 shows the GF process with an optional gasification system, the present disclosure may operate without such gasification system and may receive a C1-containing stream from an industrial or other process. The C1-containing stream may be a CO₂-containing stream having less than about 90 vol.-%, less than 85 vol.-%, less than 80 vol.-%, less than 75 vol %, less than 70 vol. % less than 65 vol.-%, less than 60 vol.-%, less than 55 vol.-%, less than 50 vol %, less than 45 vol. %, less than 40 vol.-%, less than 35 vol.-%, less than 30 vol.-%, less than 25 vol %, less than 20 vol. %, less than 15 vol.-%, less than 10 vol.-%, less than 9 vol.-%, less than 8 vol %, less than 7 vol. %, or less than 5 vol.-% which may need purification. Particularly in the case of dilute CO₂-containing streams, purification is employed so that the streams in the GF process do not contain excess inert components which would require costly larger capacity system components without an increase in product yield. Instead of the depicted gasification zone 102a, a purification zone 102b may be employed. Today's currently available CO₂ purification systems are configured and operated to achieve CO₂ purity of 99+ vol.-% or greater as is required for CO₂ sequestration operations. The purification zone 102b disclosed herein, in contrast, is configured and operated to provide a CO₂-containing stream comprising from about 90 vol.-% to about 95 vol.-% CO₂. Unlike CO₂ sequestration, the flexibility of microbial gas fermentation processes allows for lower purity CO₂ feedstock streams. Current CO₂ purification systems are not configured or operated to provide gas fermentation feedstock streams comprising from about 90 vol.-% to about 95 vol.-% CO₂.

As discussed, CO₂ is a source of carbon for GF processes, but current technology and equipment is directed to CO₂ sequestration and not to CO₂ transformation and utilization as can be accomplished through GF processes. Because GF processes are far more flexible as to the purity of CO₂ as compared to CO₂ sequestration, modifications to current CO₂ purification technology are possible, with such modifications resulting in substantially reduced energy consumption or cost to purify CO₂ feedstock for GF processes. Specifically, current technology and equipment geared for CO₂ sequestration is designed to provide a CO₂ purity of 99 vol.-% or greater. However, GF processes can successfully operate on CO₂-containing feedstock of about 90 to about 95 vol.-% CO₂. The energy required to purify a CO₂ stream above 95 vol.-%, such as from 95 vol.-% to 99 vol % or greater increase exponentially, and so significant reduction in energy consumption may be realized by purifying a CO₂ containing feedstock to about 90 vol.-% to about 95 vol.-% CO₂. In some embodiments, a CO₂ purity of from about 80 vol.-% to about 95 vol.-% CO₂ is suitable, and in other embodiments a CO₂ purity of from about 85 vol.-% to about 95 vol.-% CO₂ is suitable.

This disclosure incorporates alternative CO₂ purification techniques which have been modified as compared to their present applications to arrive at CO₂ purity in stream of from about 80, about 85, about 90 vol.-% to about 95 vol. % CO₂ which is then used as feedstock for a GF process. Although the modifications may be more costly from an equipment or operational perspective as compared to current applications of these techniques, adaptation of the technology to serve for the present purpose is less costly and requires significantly less energy that the technology currently employed for CO₂ purification, which is geared for CO₂ sequestration. By pairing suitable carbon separation techniques with GF processes, the range of suitable CO₂ sources expands greatly and allows for increased deployment of gas fermentation processes. Gases having very low CO₂ concentrations, such as flue gases, may become feedstock for gas fermentation. Key technologies appropriate for purification of CO₂-containing streams for gas fermentation include absorption, adsorption, membrane, and solid sorption separation.

In one embodiment, the CO₂ stream is purified to, for example, from about 90 vol.-% CO₂ to about 95 vol.-% CO₂ using absorption such as amine scrubbing. Amine scrubbing is a mature technology that may be modified to apply to CO₂ stream purification for gas fermentation applications. Amine scrubbing processes generally consist of two columns, a CO₂ adsorber and a CO₂ desorber. The technology uses an amine solvent to scrub CO, from a CO₂ stream such as a flue gas. The CO₂ containing gas is initially fed into an absorption column, where the solvent selectively removes the CO₂. The CO₂-rich solvent is then passed into a desorber column, where it is heated to release the CO₂. The released CO₂ is the purified CO₂ stream. The regeneration process of amine scrubbing technology is highly energy intensive, however, posing an economic and environmental challenge. As used herein, “rich” means that the outlet stream has a greater concentration of the indicated component than the inlet stream to a unit or vessel.

Aqueous monoethanolamine (MEA) is considered the benchmark amine-based solvent and has been used commercially. However, in comparison to MEA, aqueous piperazine (PZ) solutions provide better system performance by having 1) higher normalized CO₂ absorption capacity, 2) higher CO₂ absorption rate, 3) higher maximum operating temperature, 4) higher resistance to degradation, 5) lower regeneration energy demand, 6) lower volatility, and 7) lower corrosion. In addition to use of PZ solvent, the regeneration heat requirement can be further lowered by up to 45% through modifications to the standard flow scheme such as increased heat integration, rich solvent preheating, and rich solvent split feed to desorber.

While MEA may be considered the benchmark amine-based solvent, many other solvents have been identified and investigated for carbon capture such a diethanolamine (DEA), methyldiethanolamine (MDEA), diglycolamine (DGA), and 2-amino-2-methyl-1-propanol (AMP) along with PZ. Amine solvents, especially MEA, are available from a wide range of chemical suppliers in general while PZ is a more specialty solvent but is also available product from major chemical suppliers. Second-generation solvents are also available and include blends of sterically hindered alkanolamines and amino acids that require lower regeneration temperatures and are more resistant to degradation. However, these second-generation solvents may cost more than and may not perform as well as MEA.

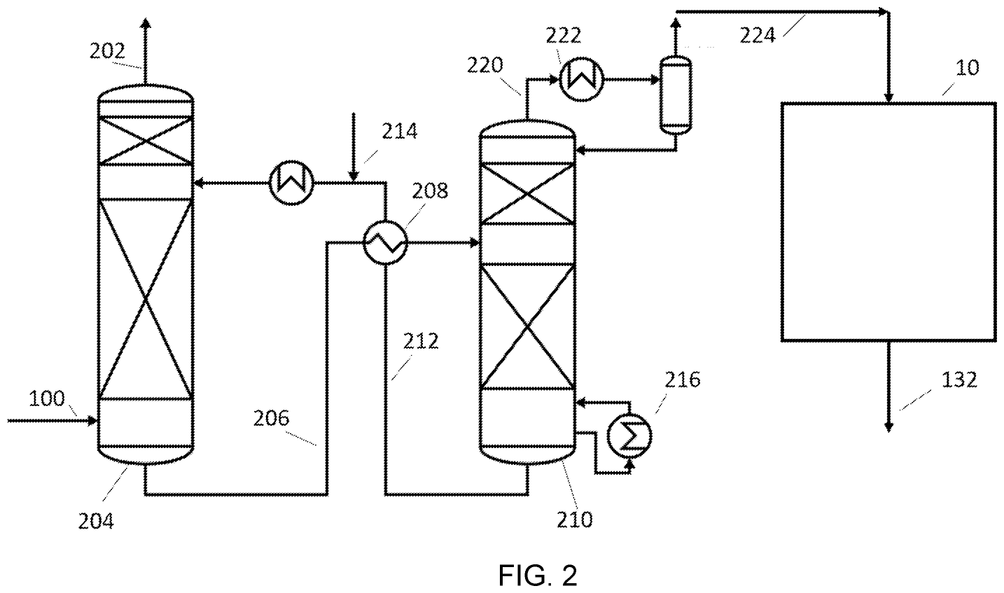

FIG. 2 shows a CO₂ stream purified to, for example, from about 90 vol.-% CO₂ to about 95 vol.-% CO₂ using amine scrubbing absorption with the resulting purified CO₂ stream passed to GF system and process such as described in FIG. 1 where the amine scrubbing absorption system is shown as 102b in FIG. 1. Feedstock 100 is passed to amine scrubber absorber column 204 containing an absorbent such as MEA. Amine scrubber absorber column 204 has vent 202. Rich solvent having absorbed CO₂ is passed as stream 206 to amine scrubber desorber column 210 after heat exchange with regenerated solvent stream 212 in heat exchanger 208. In an alternate embodiment (not shown), rich solvent 206 may be split into one or more rich solvent streams which are independently introduced into amine scrubber desorber column 210. Make-up solvent 214 is added to stream 212. Amine scrubber desorber column 210 is heated at least using bottoms reboiler 216 to a temperature where the CO₂ is desorbed from the solvent and removed in overhead 220. Overhead 220 is cooled in cooler 222 to generate cooled overhead stream 224 which is passed as feedstock comprising from about 90 vol.-% CO₂ to about 95 vol.-% CO₂ to the bioreactor and other process units of GF system 10.

In one embodiment, the CO₂ stream is purified to, for example, from about 90 vol.-% CO₂ to about 95 vol.-% CO₂ using an adsorption technique, such as a cyclic adsorption process. Advantages of cyclic adsorption processes include the availability of highly stable, easily handled, and inexpensive solid sorbents and the ability to achieve very high purities when the product is the less retained, light, component. However, major challenges arise when trying to use standard cycle configurations for the purification of CO₂ in certain applications.

Turning now to the cyclic adsorption process of pressure swing adsorption (PSA). In post-combustion gases such as flue gases, which may be primarily composed of CO₂ and N2, the target CO₂ product adsorbs more strongly on commercial adsorbents than N2 because of its higher polarizability and its quadrupole moment. This means that CO₂ is the “heavy” product in these applications and the purity of the heavy product is directly limited by the amount of non-selective gas phase contained in the column voids at the beginning of the desorption step and further decreased by the purge gas. Therefore, standard cycle configurations developed for production of high purity “light” products are not readily applicable for CO₂ purification for feedstock to a gas fermentation process.

A typical PSA cycle involves four steps: 1) feed (F) gas adsorption at high-pressure (P_{H}) with light product (LP) recovery; 2) counter-current depressurization (CnD) from P_{H} to low (vacuum) pressure (P_{L}); 3) counter-current desorption/recovery of heavy product (HP) with light reflux (LR) at P_{L} using LP gas purge; and 4) repressurization (LPP) from P_{L} to P_{H} using LP. A limitation associated with this cycle is that usage of LR necessarily causes dilution of the heavy product (HP) and modifications to the typical cycle are required to achieve high HP purity. Adding a heavy reflux (HR) step following the feed step greatly improves PSA performance. The HR step recycles a portion of HP from a P_{L} column during either the CnD or LR steps back to a P_{H} column following feed adsorption. Light components are purged from the void spaces within the column during HR which increases the purity of the HP during the collection step(s). The 4-bed 4-step HR-only cycle results in high CO₂ purity (>99%) but low recovery (15%) due to the lack of the LR purge step. Modification of the process to a 5-bed 5-step dual reflux, both LR and HR cycle, can achieve >95% CO₂ purity with much higher recovery (70-99%). Such modification of a typical PSA process is advantageous for use with a gas fermentation process.

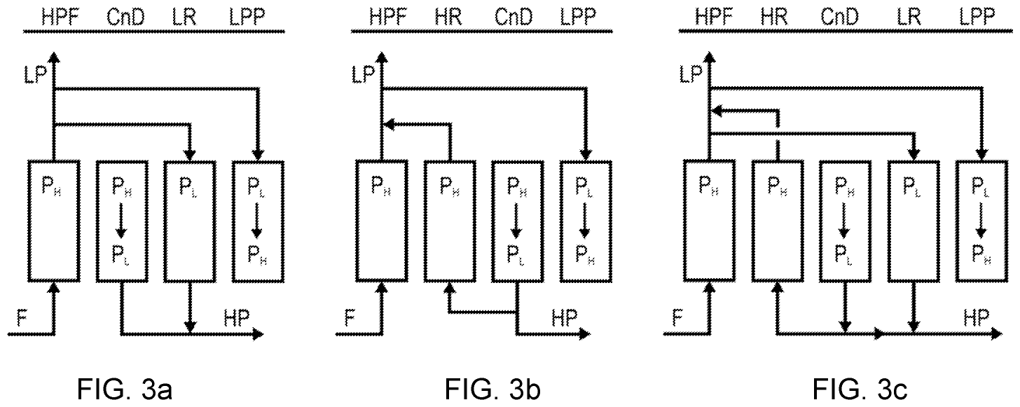

FIGS. 3a, 3b, and 3c, depict modifications of traditional PSA systems to provide feedstock comprising from about 90 vol.-% CO₂ to about 95 vol.-% CO₂ to the bioreactor and other process units of GF system 10.

A first modification of a traditional PSA system is show in FIG. 3a wherein the PSA system comprising at least four adsorbent beds wherein the pressure swing adsorption cycle comprises: a) the dilute carbon dioxide stream is passed through the first bed at a first pressure to generate a light product stream; b) the second bed is depressurized in a counter current mode from the first pressure to a second pressure lower than the first pressure generating a heavy product stream comprising carbon dioxide; c) the third bed is desorbed at the second pressure using a first portion of the light product stream to generate a second heavy product stream comprising carbon dioxide; d) the fourth bed is repressurized from the second pressure to a third pressure higher than the second pressure using a second portion of the light product stream; and e) the cycle is repeated to generate the purified carbon dioxide stream comprising from about 80 vol.-% to about 95 vol.-% carbon dioxide. The light product stream may comprise nitrogen and the third pressure may be about the same as the first pressure.

A second modification of a traditional PSA system is show in FIG. 3b wherein the PSA system comprising at least four adsorbent beds wherein the pressure swing adsorption cycle comprises: a) the dilute carbon dioxide stream is passed through the first bed at a first pressure to generate a first light product stream; b) a portion of a heavy product stream from the third bed is passed through the second bed at the first pressure in a counter current mode generating a second light product stream; c) the third bed is depressurized in a counter current mode from the first pressure to a second pressure lower than the first pressure generating the heavy product stream comprising carbon dioxide; d) the fourth bed is repressurized from the second pressure to a third pressure higher than the second pressure using a portion of the first light product stream, the second light product stream, or a combination thereof; and e) the cycle is repeated to generate the purified carbon dioxide stream comprising from about 80 vol.-% to about 95 vol.-% carbon dioxide. The light product stream may comprise nitrogen and the third pressure may be about the same as the first pressure.

A third modification of a traditional PSA system is show in FIG. 3c wherein the PSA system comprising at least five adsorbent beds wherein the pressure swing adsorption cycle comprises: a) the dilute carbon dioxide stream is passed through the first bed at a first pressure to generate a first light product stream; b) at least a portion of a first or a second heavy product stream is passed through the second bed at the first pressure in a counter current mode generating a second light product stream; c) the third bed is depressurized in a counter current mode from the first pressure to a second pressure lower than the first pressure generating the first heavy product stream comprising carbon dioxide; d) the fourth bed is desorbed at the second pressure using a first portion of the light product stream to generate a second heavy product stream comprising carbon dioxide; c) the fifth bed is repressurized from the second pressure to a third pressure higher than the second pressure using a second portion of the first light product stream, a portion of the second light product stream, or a combination thereof; and f) the cycle is repeated to generate the purified carbon dioxide stream comprising from about 80 vol.-% to about 95 vol.-% carbon dioxide.

Another cyclic adsorption technique is temperature swing adsorption (TSA). The typical TSA cycle involves three steps: 1) feed (F) gas adsorption at low temperature (TL) with light product (LP) recovery; 2) desorption/heating (H) with recovery of heavy product (HP) at high temperature (T_{H}); and 3) Cooling (C) of bed to T_{L} by indirect cooling at low-/vacuum-pressure or direct cooling with purge gas.

The typical TSA cycle faces limitations in low achievable HP purity, and even more so when inert purge gas is used. The HP purity can be increased by implementing HP recycle in a modified 5-bed 5-step cycle. The modified 5-step cycle adds an LP purge (P) step after the desorption/heating step which produces an intermediate concentration, higher than feed and lower than product, gas that is then recycled (R) into the column after the feed adsorption step. The addition of the purge and recycle steps increases the overall HP content/adsorption in the column leading to increased HP purity. Capturing the remaining desorbed HP during the purge step and recycling it also increases the overall HP recovery. A yet additional cycle modification (6-bed 6-step) implements a feed preheat step by recycling HP from the desorption/heating step directly into the feed resulting in increased HP concentration in the feed at an intermediate temperature (T_{I}). Most of the LP in the column will be desorbed during the initial heating phase so, by recycling this fraction back to the adsorption step, the HP purity can approach 100%. The 5-bed 5-step TSA cycle can achieve very high CO₂ recovery (>99%) up to 93% purity with a very steep recovery penalty as purity increases. The 6-bed 6-step cycle maintains a very similar performance curve but up to 98% purity.

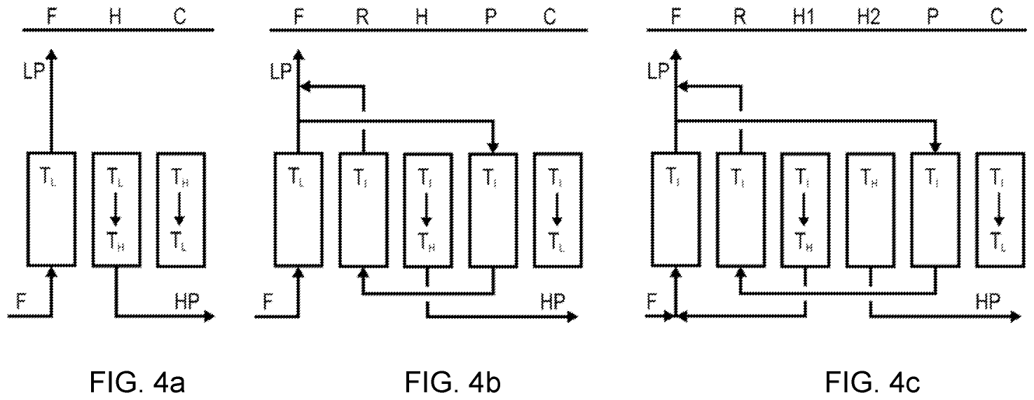

A first modification of a traditional TSA system is show in FIG. 4a wherein the PSA system comprising at least three adsorbent beds wherein the temperature swing adsorption cycle comprises: a) the dilute carbon dioxide stream is passed through the first bed at a first temperature to generate a light product stream; b) the second bed is heated in a counter current mode from the first temperature to a second temperature higher than the first temperature generating a heavy product stream comprising carbon dioxide; c) the third bed is cooled from the second temperature to a third temperature lower than the second temperature; and d) the cycle is repeated to generate the purified carbon dioxide stream comprising from about 80 vol.-% to about 95 vol.-% carbon dioxide. The light product stream may comprise nitrogen and the third temperature may be about the same as the first temperature.

A second modification of a traditional TSA system is show in FIG. 4b wherein the PSA system comprising at least five adsorbent beds wherein the temperature swing adsorption cycle comprises: a) the dilute carbon dioxide stream is passed through the first bed at a first temperature to generate a light product stream; b) an effluent from the fourth bed is passed through the second bed at a second temperature higher than the first temperature generating a second light product stream; c) the third bed is heated in a counter current mode from the second temperature to a third temperature higher than the second temperature generating a heavy product stream comprising carbon dioxide; d) at least a portion of the first or a portion of the second light product stream is passed through the fourth bed at the second temperature in a counter current mode generating the effluent stream from the fourth bed; e) the fifth bed is cooled the second temperature to a fourth temperature lower than the second temperature; and f) the cycle is repeated to generate the purified carbon dioxide stream comprising from about 80 vol.-% to about 95 vol.-% carbon dioxide. The light product stream may comprise nitrogen and the fourth temperature may be about the same as the first temperature.

A third modification of a traditional TSA system is show in FIG. 4c wherein the PSA system comprising at least five adsorbent beds wherein the temperature swing adsorption cycle comprises: a) the dilute carbon dioxide stream and an effluent from the third bed at a second temperature is mixed to preheat the dilute carbon dioxide stream to a first temperature and the mixture is passed through the first bed at the first temperature to generate a first light product stream; b) an effluent from the fifth bed is passed through the second bed in a counter current mode at the first temperature to generate a second light product stream; c) the third bed is heated in a counter current mode from the first temperature to the second temperature higher than the first temperature generating the effluent from the third bed at the second temperature; d) the fourth bed is desorbed at the second temperature to generate a heavy product stream comprising carbon dioxide; e) at least a portion of the first light product stream, a portion of the second light product stream, or both is passed through the fifth bed at the first temperature in a co current mode generating the effluent stream from the fifth bed; f) the sixth bed is cooled the second temperature to a third temperature lower than the second temperature; and g) the cycle is repeated to generate the purified carbon dioxide stream comprising from about 80 vol.-% to about 95 vol.-% carbon dioxide. The light product stream may comprise nitrogen.

In an embodiment employing TSA, the adsorbent in the adsorbent beds may be a solid sorbent and comprise at least one metal organic framework (MOF). MOFs are known in the industry and not described in detail herein. When employing one or more MOFs as the solid sorbent, the temperature swing adsorption cycle may comprise: a) passing the dilute carbon dioxide stream through a bed at a first temperature to generate a light product stream; b) heating the bed from the first temperature to a second temperature which is higher than the first temperature, for example, by direct steam injection to generate a heavy product stream comprising carbon dioxide and water; c) cooling the bed from the second temperature to a third temperature lower than the second temperature; and d) repeating the cycle to generate the purified carbon dioxide stream comprising from about 80 vol.-% to about 95 vol.-% carbon dioxide.

Referring to both PSA and TSA, Zeolite 13X is commonly selected as an adsorbent material because of its low cost and commercial availability. However, Zeolite 13X is also characterized by a higher selectivity toward water over CO₂ and low CO₂ capacity at elevated temperatures. This means that adsorption performance may be lower when separating flue gas that has not been pre-dried and pre-cooled. Avoiding an additional dehydration and/or cooling step requires selecting an adsorbent that is not sensitive to water or temperature. Hydrotalcite-like compounds (HTlcs) are adsorbents that maintain CO₂ selectivity and capacity at elevated temperatures such as 300 C and are water insensitive. HTlcs can be paired with the modified PSA and TSA cycles described above to produce high purity CO₂ without the need for additional flue gas treatment.

In one embodiment, the CO₂ stream is purified to, for example, from about 90 vol.-% CO₂ to about 95 vol.-% CO₂ using a membrane separation technique. Membrane-based CO₂ separation has some advantage over other absorption and adsorption techniques in that membranes are relatively low-cost with high modularity and relatively lower complexity. A drawback of current configuration and operation of the membrane-based separation is that separation of low-concentration and low-pressure flue gases requires membranes with high CO₂/N₂ selectivity and high power consumption to create the pressure gradient across the membranes.

Because of low CO₂ concentration in flue gases and limited CO₂/N₂ selectivity of currently available membranes, multiple membrane stages with stream recycling are desirable to achieve the target product purity and CO₂ recovery. For a multi-stage membrane with stream recycling, the retentate, such as N₂, of each stage n is recycled to the feed of stage n+1 and the permeate, such as CO₂, of each stage n is recycled to the feed of stage n−1. The effort of separation is distributed across each stage which means that increasing stage count reduces the pressure ratio across each stage, reducing overall power demand with the trade-off of higher capital cost and complexity. Both two-stage and three-stage membrane systems can meet a target CO₂ concentration of from about 80, about 85, or about 90 vol.-% to about 95 vol.-% CO₂ with up to about 90% recovery.

Membranes suitable for CO₂/N₂ separation are susceptible to temperature and water sensitivities negatively impacting performance. Polymer membranes may have a relatively low maximum operating temperature. Also, while water vapor may not directly affect polymer material performance, presence of water may reduce separation performance through competitive sorption and pore blocking. As with zeolite adsorption, performance may be lower when separating flue gas that has not been pre-dried and pre-cooled.

Suitable examples of membranes that may be used in the modified configuration for use with gas fermentation processes are described in U.S. Pat. Nos. 9,623,380, 7,896,948, and US 2023/0016870. Membrane technology is advancing, and newer membranes may be able to purify dilute CO₂ stream to the desired about 90 to about 95 vol.-% CO₂ without the need for multiple stages.

FIGS. 4a-4c illustrate an embodiment for a method of operation of an integrated gas fermentation unit. The method includes the steps of first 402 providing a gas fermentation system co-located with a carbon source (e.g., a waste carbon source). Then at step 404, fermenting at least a portion of the carbon source in a bioreactor of the gas fermentation system using at least one C1 fixing microorganism to produce a product stream. Then at step 406, integrating the product stream of the gas fermentation system with a transportation network where the transportation network is already integrated with at least another product stream from another gas fermentation system co-located with another carbon source. Finally at step 408, transporting the product stream to a remote location through the transportation network.

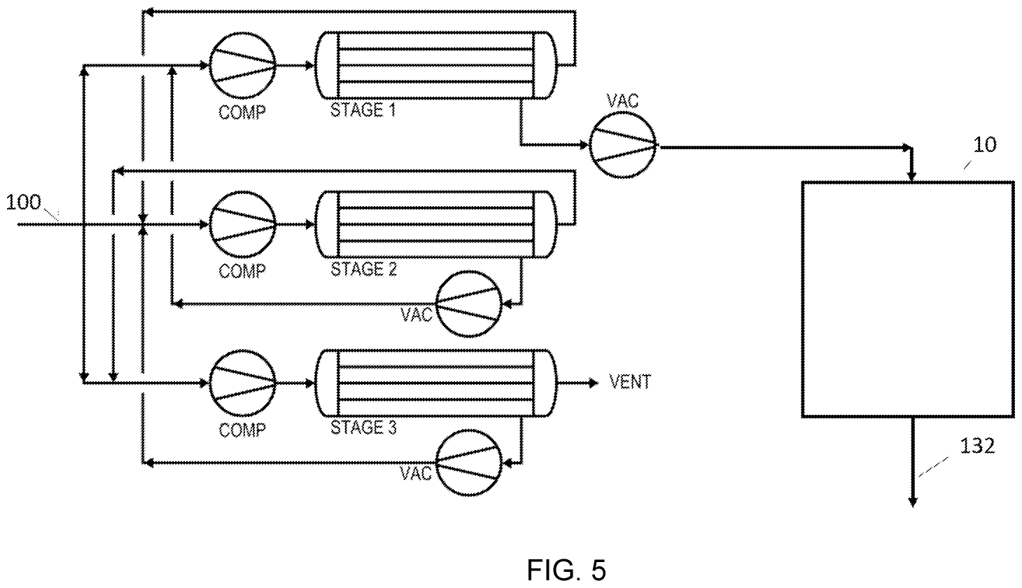

FIG. 5 illustrates an embodiment for a method of operation of an integrated gas fermentation unit. The method includes the steps of first 502 providing a carbon source from a section of the chemical manufacturing or oil refining complex 100. Then at step 504, fermenting a portion of the carbon source into a gas fermentation product with a gas fermentation unit, wherein the gas fermentation unit utilizes a microorganism to convert the portion of the carbon source into the gas fermentation product, resulting in a fermented mixture. Then at step 506, extracting the gas fermentation product from the fermented mixture. Finally at step 508, adding the gas fermentation product to a product of the chemical manufacturing or oil refining complex 100.

The product of the gas fermentation process may be further transformed by a catalytic process in a catalytic process unit which can be a device consisting of one or more vessels and/or towers or piping arrangements, which includes the continuous stirred tank reactor (CSTR), immobilized cell reactor (ICR), trickle bed reactor (TBR), bubble column, gas lift fermenter, membrane reactor such as hollow fiber membrane bioreactor (HFMBR), static mixer, or other vessel or other device suitable for gas-liquid contact. Fixed bed, moving bed, simulated moving bed, fluidized bed, entrained bed, slurry reactor, packed bed, trickle bed, batch, semi batch, continuous, plug flow, flash, dense phase, fixed bed, downflow fixed bed, upflow expanded bed, and ebullating bed.

The types of catalysts used in the catalytic process unit can include, but are not limited to, natural clays, supported or unsupported metal or metal oxide containing catalysts, acid catalysts, zeolites, organometallic compounds. Examples include activated natural or synthetic material including activated, such as acid treated, natural clays such as bentonite type of synthesized silica-alumina or silica-magnesia, optionally with added oxides of zirconium, boron or thorium; mixed metal oxides supported on alumina or silica, such as tungsten-nickel sulfide or cobalt; metal and mixed metals containing catalysts such as platinum, palladium, rhenium, rhodium, copper, nickel, optionally supported on a silica or silica-alumina base; aluminum chloride, hydrogen chloride, sulfuric acid, hydrogen fluoride, phosphates, liquid phosphoric acid, phosphoric acid on kieselguhr, copper pyrophosphate pellets, phosphoric acid film on quarts, aluminosilicates, iron, vanadium, vanadium oxide on silica, nickel, silicone dioxide, carbonic anhydrase, iodine, zeolites, silver on alumina, Ziegler-Natta catalysts, organometallic compounds, iron oxide stabilized by chromium oxide, copper, copper-zinc-alumina, promoted iron where the promoters can be potassium oxide, aluminum oxide, and calcium oxide, and iron-chrome.

The products of gas fermentation can be catalytically converted, for example, by catalytic process unit. Additionally or alternatively, the products of gas fermentation can be catalytically converted, for example, by catalytically upgrading, into molecules, or one or more second products, wherein the one or more second products are integrated into existing or newly built infrastructure or feedstock and product transportation networks. Thus, in some embodiments, molecules produced via the catalysis of the products of gas fermentation processes may also be considered desirable products or further products of fermentation. For example, in a gas fermentation system that produces ethanol, that ethanol can reacted into a range of molecules, such as propane and benzene, toluene, ethylbenzene, xylene (BTEX), and these propane and BTEX molecules can be directly introduced into the feedstock or the existing product transportation networks/pipelines.

### Ethanol and Derivatives

In one embodiment, ethanol or ethyl alcohol produced according to the method of the disclosure may be used in numerous product applications, including antiseptic hand rubs (WO 2014/100851), therapeutic treatments for methylene glycol and methanol poisoning (WO 2006/088491), as a pharmaceutical solvent for applications such as pain medication (WO 2011/034887) and oral hygiene products (U.S. Pat. No. 6,811,769), as well as an antimicrobial preservative (U.S. 2013/0230609), engine fuel (U.S. Pat. No. 1,128,549), rocket fuel (U.S. Pat. No. 3,020,708), plastics, fuel cells (U.S. Pat. No. 2,405,986), home fireplace fuels (U.S. Pat. No. 4,692,168), as an industrial chemical precursor (U.S. Pat. No. 3,102,875), cannabis solvent (WO 2015/073854), as a winterization extraction solvent (WO 2017/161387), as a paint masking product (WO 1992/008555), as a paint or tincture (U.S. Pat. No. 1,408,091), purification and extraction of DNA and RNA (WO 1997/010331), and as a cooling bath for various chemical reactions (U.S. Pat. No. 2,099,090). In addition to the foregoing, the ethanol generated by the disclosed method may be used in any other application for which ethanol might otherwise be applicable.

A further embodiment comprises converting the ethanol generated by the method into ethylene. This can be accomplished by way of an acid catalyzed dehydration of ethanol to give ethylene according to the following formula:

CH₃CH₂OH→CH₂═CH₂+H₂O

The ethylene generated in this way may be used for a variety of applications on its own or can be used as a raw material for more refined chemical products. Specifically, ethylene alone may be used as an anesthetic, as part of a mixture with nitrogen to control ripening of fruit, as a fertilizer, as an element in the production of safety glass, as part of an oxy-fuel gas in metal cutting, welding and high velocity thermal spraying, and as a refrigerant.

As a raw material, ethylene can used in the manufacture of polymers such as polyethylene (PE), polyethylene terephthalate (PET) and polyvinyl chloride (PVC) as well as fibres and other organic chemicals. These products are used in a wide variety of industrial and consumer markets such as the packaging, transportation, electrical/electronic, textile and construction industries as well as consumer chemicals, coatings and adhesives.

Ethylene can be chlorinated to ethylene dichloride (EDC) and can then be cracked to make vinyl chloride monomer (VCM). Nearly all VCM is used to make polyvinyl chloride which has its main applications in the construction industry.

Other ethylene derivatives include alpha olefins which are used in Linear low-density polyethylene (LLDPE) production, detergent alcohols and plasticizer alcohols; vinyl acetate monomer (VAM) which is used in adhesives, paints, paper coatings and barrier resins; and industrial ethanol which is used as a solvent or in the manufacture of chemical intermediates such as ethyl acetate and ethyl acrylate.

Ethylene may further be used as a monomer base for the production of various polyethylene oligomers by way of coordination polymerization using metal chloride or metal oxide catalysts. The most common catalysts consist of titanium (III) chloride, the so-called Ziegler-Natta catalysts. Another common catalyst is the Phillips catalyst, prepared by depositing chromium (VI) oxide on silica.

Polyethylene oligomers so produced may be classified according to its density and branching. Further, mechanical properties depend significantly on variables such as the extent and type of branching, the crystal structure, and the molecular weight. There are several types of polyethylene which may be generated from ethylene, including, but not limited to:
Ultra-high-molecular-weight polyethylene (UHMWPE);
Ultra-low-molecular-weight polyethylene (ULMWPE or PE-WAX);
High-molecular-weight polyethylene (HMWPE);
High-density polyethylene (HDPE);
High-density cross-linked polyethylene (HDXLPE);
Cross-linked polyethylene (PEX or XLPE);
Medium-density polyethylene (MDPE);
Linear low-density polyethylene (LLDPE);
Low-density polyethylene (LDPE);
Very-low-density polyethylene (VLDPE); and
Chlorinated polyethylene (CPE).

Low density polyethylene (LDPE) and linear low-density polyethylene (LLDPE) mainly go into film applications such as food and non-food packaging, shrink and stretch film, and non-packaging uses. High density polyethylene (HDPE) is used primarily in blow molding and injection molding applications such as containers, drums, household goods, caps and pallets. HDPE can also be extruded into pipes for water, gas and irrigation, and film for refuse sacks, carrier bags and industrial lining.

According to one embodiment, the ethylene formed from the ethanol described above may be converted to ethylene oxide via direct oxidation according to the following formula:

C₂H₄+O₂→C₂H₄O

The ethylene oxide produced thereby is a key chemical intermediate in a number of commercially important processes including the manufacture of monoethylene glycol. Other EO derivatives include ethoxylates (for use in shampoo, kitchen cleaners, etc.), glycol ethers (solvents, fuels, etc.) and ethanolamines (surfactants, personal care products, etc.).

### Monoethylene Glycol and Derivatives

According to one embodiment of the disclosure, the ethylene oxide produced as described above may be used to produce commercial quantities of monoethylene glycol by way of the formula:

(CH₂CH₂)O+H₂O→HOCH₂CH₂OH

According to another embodiment, the claimed microorganism can be modified in order to directly produce monoethylene glycol. As described in WO 2019/126400, the disclosure of which is incorporated by reference herein, the microorganism further comprises one or more of an enzymes capable of converting acetyl-CoA to pyruvate; an enzyme capable of converting pyruvate to oxaloacetate; an enzyme capable of converting pyruvate to malate; an enzyme capable of converting pyruvate to phosphoenolpyruvate; an enzyme capable of converting oxaloacetate to citryl-CoA; an enzyme capable of converting citryl-CoA to citrate; an enzyme capable of converting citrate to aconitate and aconitate to iso-citrate; an enzyme capable of converting phosphoenolpyruvate to oxaloacetate; an enzyme capable of converting phosphoenolpyruvate to 2-phospho-D-glycerate; an enzyme capable of converting 2-phospho-D-glycerate to 3-phospho-D-glycerate; an enzyme capable of converting 3-phospho-D-glycerate to 3-phosphonooxypyruvate; an enzyme capable of converting 3-phosphonooxypyruvate to 3-phospho-L-serine; an enzyme capable of converting 3-phospho-L-serine to serine; an enzyme capable of converting serine to glycine; an enzyme capable of converting 5,10-methylenetetrahydrofolate to glycine; an enzyme capable of converting serine to hydroxypyruvate; an enzyme capable of converting D-glycerate to hydroxypyruvate; an enzyme capable of converting malate to glyoxylate; an enzyme capable of converting glyoxylate to glycolate; an enzyme capable of converting hydroxypyruvate to glycolaldehyde; and/or an enzyme capable of converting glycolaldehyde to ethylene glycol.

Monoethylene glycol produced according to either of the described methods may be used as a component of a variety of products including as a raw material to make polyester fibers for textile applications, including nonwovens, cover stock for diapers, building materials, construction materials, road-building fabrics, filters, fiberfill, felts, transportation upholstery, paper and tape reinforcement, tents, rope and cordage, sails, fish netting, seatbelts, laundry bags, synthetic artery replacements, carpets, rugs, apparel, sheets and pillowcases, towels, curtains, draperies, bed ticking, and blankets.

MEG may be used on its own as a liquid coolant, antifreeze, preservative, dehydrating agent, drilling fluid or any combination thereof. The MEG produced may also be used to produce secondary products such as polyester resins for use in insulation materials, polyester film, de-icing fluids, heat transfer fluids, automotive antifreeze and other liquid coolants, preservatives, dehydrating agents, drilling fluids, water-based adhesives, latex paints and asphalt emulsions, electrolytic capacitors, paper, and synthetic leather.

Importantly, the monoethylene glycol produced may be converted to the polyester resin polyethylene terephthalate (“PET”) according to one of two major processes. The first process comprises transesterification of the monoethylene glycol utilizing dimethyl terephthalate, according to the following two-step process:

C₆H₄(CO₂CH₃)₂+2HOCH₂CH₂OH→C₆H₄(CO₂CH₂CH₂OH)₂+2CH₃OH First step

nC₆H₄(CO₂CH₂CH₂OH)₂→[(CO)C₆H₄(CO₂CH₂CH₂O)]ₙ+nHOCH₂CH₂OH Second step

Alternatively, the monoethylene glycol can be the subject of an esterification reaction utilizing terephthalic acid according to the following reaction:

nC₆H₄(CO₂H)₂+nHOCH₂CH₂OH→[(CO)C₆H₄(CO₂CH₂CH₂)]ₙ+2nH₂O

The polyethylene terephthalate produced according to either the transesterification or esterification of monoethylene glycol has significant applicability to numerous packaging applications such as jars and, in particular, in the production of bottles, including plastic bottles. It can also be used in the production of high-strength textile fibers such as Dacron, as part of durable-press blends with other fibers such as rayon, wool, and cotton, for fiber fillings used in insulated clothing, furniture, and pillows, in artificial silk, as carpet fiber, automobile tire yarns, conveyor belts and drive belts, reinforcement for fire and garden hoses, seat belts, nonwoven fabrics for stabilizing drainage ditches, culverts, and railroad beds, and nonwovens for use as diaper topsheets, and disposable medical garments.

At a higher molecular weight, PET can be made into a high-strength plastic that can be shaped by all the common methods employed with other thermoplastics. Magnetic recording tape and photographic film are produced by extrusion of PET film. Molten PET can be blow-molded into transparent containers of high strength and rigidity that are also virtually impermeable to gas and liquid. In this form, PET has become widely used in bottles, especially plastic bottles, and in jars.

### Isopropanol and Derivatives

In an additional embodiment, isopropanol or isopropyl alcohol (IPA) produced according to the method may be used in numerous product applications, including either in isolation or as a feedstock for the production for more complex products. Isopropanol may also be used in solvents for cosmetics and personal care products, de-icers, paints and resins, food, inks, adhesives, and pharmaceuticals, including products such as medicinal tablets as well as disinfectants, sterilizers, and skin creams.

The IPA produced may be used in the extraction and purification of natural products such as vegetable and animal oil and fats. Other applications include its use as a cleaning and drying agent in the manufacture of electronic parts and metals, and as an aerosol solvent in medical and veterinary products. It can also be used as a coolant in beer manufacture, a coupling agent, a polymerization modifier, a de-icing agent and a preservative.

Alternatively, the IPA produced according to the method of the disclosure may be used to manufacture additional useful compounds, including plastics, derivative ketones such as methyl isobutyl ketone (MIBK), isopropylamines and isopropyl esters. Still further, the IPA may be converted to propylene according to the following formula:

CH₃CH₂CH₂OH→CH₃—CH═CH₂

The propylene produced may be used as a monomer base for the production of various polypropylene oligomers by way of chain-growth polymerization via either gas-phase or bulk reactor systems. The most common catalysts consist of titanium (III) chloride, the so-called Ziegler-Natta catalysts and metallocene catalysts.

Polypropylene oligomers so produced may be classified according to tacticity and can be formed into numerous products by either extrusion or molding of polypropylene pellets, including piping products, heat-resistant articles such as kettles and food containers, disposable bottles (including plastic bottles), clear bags, flooring such as rugs and mats, ropes, adhesive stickers, as well as foam polypropylene which can be used in building materials. Polypropylene may also be used for hydrophilic clothing and medical dressings.

### Commodity Chemicals and Articles

According to one embodiment, the gas fermentation product is a commodity chemical. In another embodiment, the gas fermentation product is a commodity chemical, where the commodity chemical is catalytically converted, for example, by catalytically upgrading, into molecules, or one or more second products, wherein the one or more second products are integrated into existing or newly built infrastructure or feedstock and product transportation networks. In one embodiment, wherein the commodity chemical is selected from ethanol, isopropanol, monoethylene glycol, sulfuric acid, propylene, sodium hydroxide, sodium carbonate, ammonia, benzene, acetic acid, ethylene, ethylene oxide, formaldehyde, methanol, or any combination thereof. In one embodiment, the commodity chemical is aluminum sulfate, ammonia, ammonium nitrate, ammonium sulfate, carbon black, chlorine, diammonium phosphate, monoammonium phosphate, hydrochloric acid, hydrogen fluoride, hydrogen peroxide, nitric acid, oxygen, phosphoric acid, sodium silicate, titanium dioxide, or any combination thereof. In another embodiment, the commodity chemical is acetic acid, acetone, acrylic acid, acrylonitrile, adipic acid, benzene, butadiene, butanol, caprolactam, cumene, cyclohexane, dioctyl phthalate, ethylene glycol, methanol, octanol, phenol, phthalic anhydride, polypropylene, polystyrene, polyvinyl chloride, polypropylene glycol, propylene oxide, styrene, terephthalic acid, toluene, toluene diisocyanate, urea, vinyl chloride, xylenes, or any combination thereof.

### Secondary Products

The disclosed systems and methods are also suitable for providing one or more secondary products that are independent of the gas fermentation product (e.g., ethylene, ethanol, acetate, etc.). For example, in certain embodiments, microbial biomass itself may be considered a secondary product. In such embodiments, biomass from a bioreactor, such as dead microorganisms, may be used as a carbon source for further fermentation by gasifying the biomass. Additionally or alternatively, microbial proteins or other biomass may be recovered from a bioreactor and sold/used separately from the primary product (e.g., ethylene, ethanol, acetate, 1-butanol, etc.) as a supplement, such as a nutritional supplement and/or an animal feed. Known methods for using such biomass as a nutritional supplement or animal feed are disclosed in U.S. Pat. No. 10,856,560, which is herein incorporated by reference.

Additionally or alternatively, biochar may be a secondary product. In embodiments that involve or comprise gasification of solid or liquid carbonaceous materials to produce a feedstock, biochar can be incidentally produced. Biochar is carbon rich and highly structured, and therefore it can be useful as, for example, fertilizer, among other applications.

Additionally or alternatively, unutilized carbon dioxide, which may be in the form of an off-gas from the gas fermentation, may be a secondary product. Such unutilized carbon dioxide will be in a stoichiometrically higher proportion in the off-gas compared to the feedstock, and this relative purity can make the carbon dioxide useful. For example, the unutilized carbon can be sequestered by an operator for the purposes of obtaining carbon credits, or it may be combined with hydrogen gas (H₂), such as “green hydrogen” resulting from electrolysis, and recycled back into the gas fermenter or bioreactor as feedstock.

### C. Microorganisms and Fermentation

The disclosed systems and methods integrate microbial fermentation into existing or newly built infrastructure of, for example, a gas (e.g., natural gas) transportation pipeline, oil well, or the like to convert various feedstocks, gas, or other by-products into useful products such as ethylene. As disclosed herein, the systems allow for feedstocks, gas, or other by-products to be directly provided to a bioreactor, and the bioreactor is directly connected to a system for facilitating transport of a desirable product of fermentation to an end point (e.g., a chemical plant or refinery). In particular, the disclosed systems and methods are applicable for producing useful products (e.g., ethylene, ethanol, acetate, etc.) from gaseous substrates, such as gases that may optionally contain H₂, that are utilized as a carbon source by microbial cultures. Such microorganisms may include bacteria, archaea, algae, or fungi (e.g., yeast), and these classes of microorganism may be suitable for the disclosed systems and methods. In general, the selection of the microorganism(s) is not particularly limited so long as the microorganism is C1-fixing, carboxydotrophic, acetogenic, methanogenic, capable of Wood-Ljungdahl synthesis, a hydrogen oxidizer, autotrophic, chemolithoautotrophic, or any combination thereof. Among the various suitable classes of microorganisms, bacteria are particularly well suited for integration in the disclosed systems and methods.

When bacteria are utilized in the disclosed systems and methods, the bacteria may be aerobic or anaerobic, depending on the nature of the carbon source and other inputs being fed into the bioreactor or fermentation unit. Further, the bacteria utilized in the disclosed systems and methods can include one of more strains of carboxydotrophic bacteria. In particular embodiments, the carboxydotrophic bacterium can be selected from a genus including, but not limited to, Cupriavidus, Clostridium, Moorella, Carboxydothermus, Ruminococcus, Acetobacterium, Eubacterium, Butyribacterium, Oxobacter, Methanosarcina, Methanosarcina, and Desulfotomaculum. In particular embodiments, the carboxydotrophic bacterium is Clostridium autoethanogenum. In other particular embodiments, the carboxydotrophic bacterium is Cupriavidus necator.

A number of anaerobic bacteria are known to be capable of carrying out fermentation for the disclosed methods and system. Examples of such bacteria that are suitable for use in the disclosure include bacteria of the genus Clostridium, such as strains of Clostridium ljungdahlii (including those described in WO 00/68407, EP 117309, U.S. Pat. Nos. 5,173,429, 5,593,886, and 6,368,819, WO 98/00558 and WO 02/08438), Clostridium carboxydivorans (Liou et al., International Journal of Systematic and Evolutionary Microbiology 33: pp 2085-2091) and Clostridium autoethanogenum (Abrini et al., Archives of Microbiology 161: pp 345-351). Other suitable bacteria include those of the genus Moorella, including Moorella sp HUC22-1 (Sakai et al., Biotechnology Letters 29: pp 1607-1612), and those of the genus Carboxydothermus (Svetlichny, V. A., et al. (1991), Systematic and Applied Microbiology 14:254-260). The disclosures of each of these publications are incorporated herein by reference. In addition, other carboxydotrophic anaerobic bacteria can be used in the disclosed systems and methods by a person of skill in the art. It will also be appreciated upon consideration of the instant disclosure that a mixed culture of two or more bacteria may be used in the disclosed systems and methods. All of the foregoing patents, patent applications, and non-patent literature are incorporated herein by reference in their entirety.

One exemplary anaerobic bacteria that is suitable for use in the disclosed systems and methods is Clostridium autoethanogenum. In some embodiments, the Clostridium autoethanogenum is a Clostridium autoethanogenum having the identifying characteristics of the strain deposited at the German Resource Centre for Biological Material (DSMZ) under the identifying deposit number 19630. In some embodiments, the Clostridium autoethanogenum is a Clostridium autoethanogenum having the identifying characteristics of DSMZ deposit number DSMZ 10061. In some embodiments, the Clostridium autoethanogenum is a Clostridium autoethanogenum having the identifying characteristics of DSMZ deposit number DSMZ 23693.

In some embodiments, the anaerobic bacteria is Clostridium carboxidivorans having the identifying characteristics of deposit number DSM15243. In some embodiments, the anaerobic bacteria is Clostridium drakei having the identifying characteristics of deposit number DSM12750. In some embodiments, the anaerobic bacteria is Clostridium ljungdahlii having the identifying characteristics of deposit number DSM13528. Other suitable Clostridium ljungdahlii strains may include those described in WO 00/68407, EP 117309, U.S. Pat. Nos. 5,173,429, 5,593,886, and 6,368,819, WO 98/00558 and WO 02/08438, all of which are incorporated herein by reference. In some embodiments, the anaerobic bacteria is Clostridium scatologenes having the identifying characteristics of deposit number DSM757. In some embodiments, the anaerobic bacteria is Clostridium ragsdalei having the identifying characteristics of deposit number ATCC BAA-622.

In some embodiments, the anaerobic bacteria is Acetobacterium woodii. In some embodiments, the anaerobic bacteria is from the genus Moorella, such as Moorella sp HUC22-1, (Sakai et al, Biotechnology Letters, 29: pp 1607-1612). Further examples of suitable anaerobic bacteria include, but are not limited to, Morella thermoacetica, Moorella thermoautotrophica, Ruminococcus productus, Acetobacterium woodii, Eubacterium limosum, Butyribacterium methylotrophicum, Oxobacter pfennigii, Methanosarcina barkeri, Methanosarcina acetivorans, Desulfotomaculum kuznetsovii (Simpa et. al. Critical Reviews in Biotechnology, 2006 Vol. 26. Pp 41-65). In addition, it should be understood that other C1-fixing, carboxydotrophic anaerobes may be suitable for the disclosed systems and methods. It will also be appreciated that a mixed culture of two or more bacteria may be utilized as well.

A number of aerobic bacteria are known to be capable of carrying out fermentation for the disclosed methods and system. Examples of such bacteria that are suitable for use in the disclosure include bacteria of the genus Cupriavidus and Ralstonia. In some embodiments, the aerobic bacteria is Cupriavidus necator or Ralstonia eutropha. In some embodiments, the aerobic bacteria is Cupriavidus alkaliphilus. In some embodiments, the aerobic bacteria is Cupriavidus basilensis. In some embodiments, the aerobic bacteria is Cupriavidus campinensis. In some embodiments, the aerobic bacteria is Cupriavidus gilardii. In some embodiments, the aerobic bacteria is Cupriavidus laharis. In some embodiments, the aerobic bacteria is Cupriavidus metallidurans. In some embodiments, the aerobic bacteria is Cupriavidus nantongensis. In some embodiments, the aerobic bacteria is Cupriavidus numazuensis. In some embodiments, the aerobic bacteria is Cupriavidus oxalaticus. In some embodiments, the aerobic bacteria is Cupriavidus pampae. In some embodiments, the aerobic bacteria is Cupriavidus pauculus. In some embodiments, the aerobic bacteria is Cupriavidus pinatubonensis. In some embodiments, the aerobic bacteria is Cupriavidus plantarum. In some embodiments, the aerobic bacteria is Cupriavidus respiraculi. In some embodiments, the aerobic bacteria is Cupriavidus taiwanensis. In some embodiments, the aerobic bacteria is Cupriavidus yeoncheonensis.

The fermentation may be carried out in any suitable bioreactor. In some embodiments, the bioreactor may comprise a first, growth reactor in which the microorganisms (e.g., bacteria) are cultured, and a second, fermentation reactor, to which fermentation broth from the growth reactor is fed and in which most of the fermentation product (e.g. ethylene, ethanol, acetate, etc.) is produced.

It will be appreciated that for growth of the bacteria and fermentation to occur, in addition to a carbon-containing substrate gas, a suitable liquid nutrient medium will need to be fed to the bioreactor. A nutrient medium will contain vitamins and minerals sufficient to permit growth of the micro-organism used. Aerobic and anaerobic media suitable for the fermentation using carbon-containing substrate gases as the sole carbon source are known in the art. For example, suitable media are described in U.S. Pat. Nos. 5,173,429 and 5,593,886 and WO 02/08438, WO2007/115157 and WO2008/115080, referred to above and all of which are incorporated herein by reference. Further, the fermentation can be carried out under appropriate conditions for the desired fermentation to occur. Reaction conditions that should be considered include pressure, temperature, gas flow rate, liquid flow rate, media pH, media redox potential, agitation rate (if using a continuous stirred tank reactor), inoculum level, maximum gas substrate concentrations, and maximum product concentrations to avoid product inhibition.

The optimum reaction conditions will depend partly on the particular micro-organism used. However, in general, it may be preferable that the fermentation be performed at a pressure higher than ambient pressure. Operating at increased pressures may allow for, for example, a significant increase in the rate of CO transfer from the gas phase to the liquid phase where it can be taken up by the micro-organism as a carbon source. This, in turn, means that the retention time (defined as the liquid volume in the bioreactor divided by the input gas flow rate) can be reduced when bioreactors are maintained at elevated pressure rather than atmospheric pressure. Also, since a given CO, or CO₂ and H₂ conversion rate is in part a function of the substrate retention time, and achieving a desired retention time in turn dictates the required volume of a bioreactor, the use of pressurized systems can greatly reduce the volume of the bioreactor required, and consequently the capital cost of the fermentation equipment.

Similarly, temperature of the culture may vary as needed. For example, in some embodiments, the fermentation is carried out at a temperature of about 34° C. to about 37° C. In some embodiments, the fermentation is carried out at a temperature of about 34° C. This temperature range may assist in supporting or increasing the efficiency of fermentation including, for example, maintaining or increasing the growth rate of bacteria, extending the period of growth of bacteria, maintaining or increasing production of the desired product (e.g., ethylene, ethanol, acetate, etc.), or maintaining or increasing CO or CO₂ uptake or consumption.

Culturing of the bacteria used in in the disclosed systems and methods may be conducted using any number of processes known in the art for culturing and fermenting substrates. In some embodiments a culture of a bacterium can be maintained in an aqueous culture medium. For example, the aqueous culture medium may be a minimal anaerobic microbial growth medium. Suitable media are known in the art and described for example in U.S. Pat. Nos. 5,173,429 and 5,593,886; WO 02/08438, and in Klasson et al (1992), Bioconversion of Synthesis Gas into Liquid or Gaseous Fuels, Enz. Microb. Technol. 14:602-608; Najafpour and Younesi (2006) Ethanol and acetate synthesis from waste gas using batch culture of Clostridium ljungdahlii. Enzyme and Microbial Technology, 38 (1-2): 223-228; and Lewis et al (2002), Making the connection-conversion of biomass-generated producer gas to ethanol, Abst. Bioenergy, p. 2091-2094.

Further general processes for using gaseous substrates for fermentation that may be utilized for the disclosed systems and methods are described in the following disclosures: WO98/00558, M. Demler and D. Weuster-Botz (2010), Reaction Engineering Analysis of Hydrogenotrophic Production of Acetic Acid by Acetobacterium woodii, Biotechnology and Bioengineering; D. R. Martin, A. Misra and H. L. Drake (1985), Dissimilation of Carbon Monoxide to Acetic Acid by Glucose-Limited Cultures of Clostridium thermoaceticum, Applied and Environmental Microbiology, 49 (6): 1412-1417. Further processes generally described in the following articles using gaseous substrates for fermentation may also be utilized: (i) K. T. Klasson, et al. (1991), Bioreactors for synthesis gas fermentations resources, Conservation and Recycling, 5:145-165; (ii) K. T. Klasson, et al. (1991), Bioreactor design for synthesis gas fermentations, Fuel, 70:605-614; (iii) K. T. Klasson, et al. (1992), Bioconversion of synthesis gas into liquid or gaseous fuels, Enzyme and Microbial Technology, 14:602-608; (iv) J. L. Vega, et al. (1989), Study of Gaseous Substrate Fermentation: Carbon Monoxide Conversion to Acetate. 2. Continuous Culture, Biotech. Bioeng., 34 (6): 785-793; (vi) J. L. Vega, et al. (1989), Study of gaseous substrate fermentations: Carbon monoxide conversion to acetate. 1. Batch culture, Biotech. Bioeng., 34 (6): 774-784; (vii) J. L. Vega, et al. (1990), Design of Bioreactors for Coal Synthesis Gas Fermentations, Resources, Conservation and Recycling, 3:149-160; all of which are incorporated herein by reference.

As noted above, while bacteria may be used for illustration of microorganisms for the disclosed systems and methods, other microorganisms like yeast may also be suitable. For example, yeast that may be used in the disclosed systems and methods include genus Cryptococcus, such as strains of Cryptococcus curvatus (also known as Candida curvatus) (see Chi et al. (2011), Oleaginous yeast Cryptococcus curvatus culture with dark fermentation hydrogen production effluent as feedstock for microbial lipid production, International Journal of Hydrogen Energy, 36:9542-9550, which is incorporated herein by reference). Other suitable yeasts include those of the genera Candida, Lipomyces, Rhodosporidium, Rhodotorula, Saccharomyces, and Yarrowia. In addition, it should be understood that the disclosed systems and methods may utilize a mixed culture of two or more yeasts. Additional fungi that may be suitable for the disclosed systems and methods include, but are not limited to, fungi selected from Blakeslea, Cryptococcus, Cunninghamella, Mortierella, Mucor, Phycomyces, Pythium, Thraustochytrium and Trichosporon. Culturing of yeast or other fungi may be conducted using any number of processes known in the art for culturing and fermenting substrates using yeasts or fungi.

Typically, fermentation is carried out in any suitable bioreactor, such as a continuous stirred tank reactor (CTSR), a bubble column reactor (BCR) or a trickle bed reactor (TBR). Also, in some embodiments, the bioreactor may comprise a first, growth reactor in which the micro-organisms are cultured, and a second, fermentation reactor, to which fermentation broth from the growth reactor is fed and in which most of the fermentation product (e.g., ethylene, ethanol, acetate, etc.) is produced.

The disclosed systems and method may comprise a primary bioreactor and a secondary bioreactor. The efficiency of the fermentation processes may be further improved by a further process of recycling a stream exiting the secondary bioreactor to at least one primary reactor. The stream exiting the secondary bioreactor may contain unused substrates, salts, and other nutrient components. By recycling the exit stream to a primary reactor, the cost of providing a continuous nutrient media to the primary reactor can be reduced. This recycling step has the further benefit of potentially reducing the water requirements of the continuous fermentation process. The stream exiting the bioreactor can optionally be treated before being passed back to a primary reactor. For example, because yeasts generally require oxygen for growth, any media recycled from a secondary bioreactor to a primary bioreactor may need to have all oxygen substantially removed, as any oxygen present in the primary bioreactor will be harmful to an anaerobic culture in the primary bioreactor. Therefore, the broth stream exiting the secondary bioreactor may be passed through an oxygen scrubber to remove substantially all of the oxygen prior to being passed to the primary reactor. In some embodiments, biomass from a bioreactor (e.g., a primary bioreactor, secondary bioreactor, or any combination thereof) may be separated and processed to recover one or more products.

In some embodiments, both anaerobic and aerobic gases can be used to feed separate cultures (e.g., an anaerobic culture and an aerobic culture) in two or more different bioreactors that are both integrated into the same process stream.

As disclosed herein, the feedstock gas stream providing a carbon source for the disclosed cultures is not particularly limited, so long as it contains a carbon source. C1 feedstocks comprising methane, carbon monoxide, carbon dioxide, or any combination thereof may be employed. Optionally, H₂ may also be present in the feedstock. In some embodiments, the feedstock may comprise a gaseous substrates comprising substrate comprising carbon monoxide. In some embodiments, the feedstock may comprise a gaseous substrates comprising substrate comprising carbon dioxide. In some embodiments, the feedstock may comprise a gaseous substrates comprising substrate comprising both carbon monoxide and carbon dioxide. In some embodiments, the feedstock may comprise a gaseous substrates comprising carbon monoxide. In some embodiments, the feedstock may comprise a gaseous substrates comprising carbon dioxide. In some embodiments, the feedstock may comprise a gaseous substrates comprising carbon monoxide, carbon dioxide, or any combination thereof.

Regardless of the source or precise content of the gas used as a feedstock, the feedstock may be metered (e.g., for carbon credit calculations or mass balancing of sustainable carbon with overall products) into a bioreactor in order to maintain control of the follow rate and amount of carbon provided to the culture. Similarly, the output of the bioreactor may be metered (e.g., for carbon credit calculations or mass balancing of sustainable carbon with overall products) or comprise a valved connection that can control the flow of the output and products (e.g., ethylene, ethanol, acetate, 1-butanol, etc.) produced via fermentation. Such a valve or metering mechanism can be useful for a variety of purposes including, but not limited to, slugging of product through a connected pipeline and measuring the amount of output from a given bioreactor such that if the product is mixed with other gases or liquids the resulting mixture can later be mass balanced to determine the percentage of the product that was produced from the bioreactor.

The microorganisms of the disclosure may be cultured with the gaseous substrate to produce one or more products. For instance, the microorganism may produce or may be engineered to produce ethanol (WO 2007/117157, U.S. Pat. No. 7,972,824), acetate (WO 2007/117157, U.S. Pat. No. 7,972,824), 1-butanol (WO 2008/115080, U.S. Pat. No. 8,293,509, WO 2012/053905, U.S. Pat. No. 9,359,611 and WO 2017/066498, U.S. Pat. No. 9,738,875), butyrate (WO 2008/115080, U.S. Pat. No. 8,293,509), 2,3-butanediol (WO 2009/151342, U.S. Pat. No. 8,658,408 and WO 2016/094334, U.S. Pat. No. 10,590,406), lactate (WO 2011/112103, U.S. Pat. No. 8,900,836), butene (WO 2012/024522, US2012/045807), butadiene (WO 2012/024522, US 2012/045807), methyl ethyl ketone (2-butanone) (WO 2012/024522, US 2012/045807 and WO 2013/185123, U.S. Pat. No. 9,890,384), ethanol which is then converted to ethylene (WO 2012/026833, US 2013/157,322), acetone (WO 2012/115527, U.S. Pat. No. 9,410,130), isopropanol (WO 2012/115527 U.S. Pat. No. 9,410,130), lipids (WO 2013/036147 U.S. Pat. No. 9,068,202), 3-hydroxypropionate (3-HP) (WO 2013/180581, U.S. Pat. No. 9,994,878), terpenes, including isoprene (WO 2013/180584, U.S. Pat. No. 10,913,958), fatty acids (WO 2013/191567 U.S. Pat. No. 9,347,076), 2-butanol (WO 2013/185123 U.S. Pat. No. 9,890,384), 1,2-propanediol (WO 2014/036152, U.S. Pat. No. 9,284,564), 1-propanol (WO 2014/0369152, U.S. Pat. No. 9,284,564), 1 hexanol (WO 2017/066498, U.S. Pat. No. 9,738,875), 1 octanol (WO 2017/066498, U.S. Pat. No. 9,738,875), chorismate-derived products (WO 2016/191625, U.S. Pat. No. 10,174,303), 3-hydroxybutyrate (WO 2017/066498, U.S. Pat. No. 9,738,875), 1,3-butanediol (WO 2017/066498, U.S. Pat. No. 9,738,875), 2-hydroxyisobutyrate or 2-hydroxyisobutyric acid (WO 2017/066498, U.S. Pat. No. 9,738,875), isobutylene (WO 2017/066498, U.S. Pat. No. 9,738,875), adipic acid (WO 2017/066498, U.S. Pat. No. 9,738,875), 1,3-hexanediol (WO 2017/066498, U.S. Pat. No. 9,738,875), 3-methyl-2-butanol (WO 2017/066498, U.S. Pat. No. 9,738,875), 2-buten-1-ol (WO 2017/066498, U.S. Pat. No. 9,738,875), isovalerate (WO 2017/066498, U.S. Pat. No. 9,738,875), isoamyl alcohol (WO 2017/066498, U.S. Pat. No. 9,738,875), and/or monoethylene glycol (WO 2019/126400, U.S. Pat. No. 11,555,209) in addition to 2-phenylethanol (WO 2021/188190, US 2021/0292732) and ethylene.

### Substrates and C1-Carbon Sources

The substrate and/or C1-carbon source may be a gas obtained as a by-product of an industrial process or from another source, such as combustion engine exhaust fumes, biogas, landfill gas, direct air capture, flaring, or from electrolysis. The substrate and/or C1-carbon source may be syngas generated by pyrolysis, torrefaction, or gasification. In other words, carbon in solid or liquid materials may be recycled by pyrolysis, torrefaction, or gasification to generate syngas which is used as the substrate and/or C1-carbon source in gas fermentation. The substrate and/or C1-carbon source may be natural gas. The substrate and/or C1-carbon source carbon dioxide from conventional and unconventional gas production. The substrate and/or C1-carbon source may be a gas comprising methane. Gas fermentation processes are flexible and any of these substrate and/or C1-carbon sources may be employed.

In certain embodiments, the industrial process source of the substrate and/or C1 carbon source is selected from ferrous metal products manufacturing, such as a steel manufacturing, non-ferrous products manufacturing, petroleum refining, electric power production, carbon black production, paper and pulp manufacturing, ammonia production, methanol production, coke manufacturing, petrochemical production, carbohydrate fermentation, cement making, aerobic digestion, anaerobic digestion, catalytic processes, natural gas extraction, cellulosic fermentation, oil extraction, industrial processing of geological reservoirs, processing fossil resources such as natural gas coal and oil, landfill operations, or any combination thereof. Examples of specific processing steps within an industrial process include catalyst regeneration, fluid catalyst cracking, and catalyst regeneration. Air separation and direct air capture are other suitable industrial processes. Specific examples in steel and ferroalloy manufacturing include blast furnace gas, basic oxygen furnace gas, coke oven gas, direct reduction of iron furnace top-gas, and residual gas from smelting iron. Other general examples include flue gas from fired boilers and fired heaters, such as natural gas, oil, or coal fired boilers or heaters, and gas turbine exhaust. Another example is the flaring of compounds such as at oil and gas production sites. In these embodiments, the substrate and/or C1-carbon source may be captured from the industrial process before it is emitted into the atmosphere, using any known method.

The substrate and/or C1-carbon source may be synthesis gas known as syngas, which may be obtained from reforming, partial oxidation, plasma, or gasification processes. Examples of gasification processes include gasification of coal, gasification of refinery residues, gasification of petroleum coke, gasification of biomass, gasification of lignocellulosic material, gasification of wood, gasification of black liquor, gasification of municipal solid waste, gasification of municipal liquid waste, gasification of industrial solid waste, gasification of industrial liquid waste, gasification of refuse derived fuel, gasification of sewerage, gasification of sewerage sludge, gasification of sludge from wastewater treatment, gasification of landfill gas, gasification of biogas such as when biogas is added to enhance gasification of another material. Examples of reforming processes include, steam methane reforming, steam naphtha reforming, reforming of natural gas, reforming of biogas, reforming of landfill gas, reforming of coke oven gas, reforming of pyrolysis off-gas, reforming of ethylene production off-gas, naphtha reforming, and dry methane reforming. Examples of partial oxidation processes include thermal and catalytic partial oxidation processes, catalytic partial oxidation of natural gas, partial oxidation of hydrocarbons, partial oxidation of biogas, partial oxidation of landfill gas, or partial oxidation of pyrolysis off-gas. Examples of municipal solid waste include tires, plastics, refuse derived fuel, and fibers such as in shoes, apparel, and textiles. Municipal solid waste may be simply landfill-type waste and may be sorted or unsorted. Examples of biomass may include lignocellulosic material and microbial biomass. Lignocellulosic material may include agriculture by-products, forest by-products, and some industrial by-products.

Biomass may be created as by-products of “nature-based solutions” (NBS) and thus natured-based solutions may provide feedstock to the gas fermentation process. Nature-based solutions is articulated by the European Commission as solutions inspired and supported by nature, which are cost-effective, simultaneously provide environmental, social, and economic benefits and help build resilience. Such solutions bring more, and more diverse, nature and natural features and processes into cities, landscapes, and seascapes, through locally adapted, resource-efficient, and systemic interventions. Nature-based solutions must benefit biodiversity and support the delivery of a range of ecosystem services. Through the use of NBS healthy, resilient, and diverse ecosystems (whether natural, managed, or newly created) can provide solutions for the benefit of both societies and overall biodiversity. Examples of nature-based solutions include natural climate solutions (conservation, restoration and improved land management that increase carbon storage or avoid greenhouse gas emissions in landscapes and wetlands across the globe), halting biodiversity loss, socio-economic impact efforts, habitat restoration, and health and wellness efforts with respect to air and water. Biomass produced through nature-based solutions may be used as feedstock to gas fermentation processes.

As shown, the optional step of a gasification process in the overall gas fermentation process greatly increases suitable feedstocks to the overall gas fermentation process as compared to gaseous feedstocks alone. Further, incentives achieved may extend beyond items such as carbon credits, and into the natural based solutions space.

The substrate and/or C1-carbon source may be a gas stream comprising methane. Such a methane containing gas may be obtained from: fossil methane emissions such as during fracking, wastewater treatment, livestock, agriculture, and municipal solid waste landfills. It is also envisioned that the methane may be burned to produce electricity or heat and the C1 by-products may be used as the substrate or carbon source. The substrate and/or C1-carbon source may be a gas stream comprising natural gas.
Embodiment 1. A method of carbon utilization comprising: passing a dilute carbon dioxide stream to a purification zone wherein the dilute carbon dioxide stream comprises less than about 80 vol.-% carbon dioxide; purifying the dilute carbon dioxide stream to generate a purified carbon dioxide stream comprising from about 80 vol.-% to about 95 vol.-% carbon dioxide, wherein the purifying is by a process selected from an amine absorption process, a temperature swing adsorption process, a pressure swing adsorption process, a membrane separation process, or any combination thereof; passing hydrogen and the purified carbon dioxide stream to a gas fermentation zone and contacting with C1-fixing microorganism biocatalyst to ferment at least the hydrogen and the purified carbon dioxide stream and produce at least one target product; and recovering the target product.
Embodiment 2. The method of embodiment 1 wherein the dilute carbon dioxide stream comprises less than about 90 vol.-% carbon dioxide, and the purified carbon dioxide stream comprises from about 90 vol.-% to about 95 vol.-% carbon dioxide.
Embodiment 3. The method of any of embodiments 1 or 2 wherein the C1-fixing microorganism is an acetogenic carboxydotrophic microorganism.
Embodiment 4. The method of any of embodiments 1 to 3 wherein the purification process is amine absorption and the solvent of the amine absorption process is selected from aqueous solutions of monoethanolamine, piperazine, diethanolamine, methyldiethanolamine, diglycolamine, 2-amino-2-methyl-1-propanol, or any combination thereof.
Embodiment 5. The method of embodiment 4, or any of the preceding embodiments, wherein the amine absorption purification process comprises an absorber column and a desorber column, wherein a rich solvent from the absorber column is preheated by heat exchange with regenerated solvent from the desorber column.
Embodiment 6. The method of any of embodiments 4 or 5, or any of the preceding embodiments, wherein the amine absorption purification process comprises an absorber column and a desorber column, and a rich solvent from the absorber column is split into two or more rich solvent streams and each rich solvent stream is passed to the desorber column.
Embodiment 7. The method of any of embodiments 1 to 3, or any of the preceding embodiments, wherein the purification process is pressure swing adsorption comprising at least four adsorbent beds wherein the pressure swing adsorption cycle comprises: passing the dilute carbon dioxide stream through the first bed at a first pressure to generate a light product stream; depressurizing the second bed in a counter current mode from the first pressure to a second pressure lower than the first pressure generating a heavy product stream comprising carbon dioxide; desorbing the third bed at the second pressure using a first portion of the light product stream to generate a second heavy product stream comprising carbon dioxide; repressurizing the fourth bed from the second pressure to a third pressure higher than the second pressure using a second portion of the light product stream; and repeating the cycle is to generate the purified carbon dioxide stream comprising from about 80 vol.-% to about 95 vol.-% carbon dioxide.
Embodiment 8. The method of embodiment 7, or any of the preceding embodiments, wherein the light product stream comprises nitrogen.
Embodiment 9. The method of embodiment 7 or 8, or any of the preceding embodiments, wherein the third pressure is about the same as the first pressure.
Embodiment 10. The method of any of embodiments 1 to 3, or any of the preceding embodiments, wherein the purification process is pressure swing adsorption comprising at least four adsorbent beds wherein the pressure swing adsorption cycle comprises: passing the dilute carbon dioxide stream through the first bed at a first pressure to generate a first light product stream; passing a portion of a heavy product stream from the third bed through the second bed at the first pressure in a counter current mode generating a second light product stream; depressurizing the third bed in a counter current mode from the first pressure to a second pressure lower than the first pressure generating the heavy product stream comprising carbon dioxide; repressurizing the fourth bed from the second pressure to a third pressure higher than the second pressure using a portion of the first light product stream, the second light product stream, or a combination thereof; and repeating the cycle to generate the purified carbon dioxide stream comprising from about 80 vol.-% to about 95 vol.-% carbon dioxide.
Embodiment 11. The method of embodiment 10, or any of the preceding embodiments, wherein the light product stream comprises nitrogen.
Embodiment 12. The method of embodiment 10 or 11, or any of the preceding embodiments, wherein the third pressure is about the same as the first pressure.
Embodiment 13. The method of any of embodiments 1 to 3, or any of the preceding embodiments, wherein the purification process is pressure swing adsorption comprising at least five adsorbent beds wherein the pressure swing adsorption cycle comprises: passing the dilute carbon dioxide stream through the first bed at a first pressure to generate a first light product stream; passing at least a portion of a first or a second heavy product stream is through the second bed at the first pressure in a counter current mode generating a second light product stream; depressurizing the third bed in a counter current mode from the first pressure to a second pressure lower than the first pressure to generate the first heavy product stream comprising carbon dioxide; desorbing fourth bed at the second pressure using a first portion of the light product stream to generate a second heavy product stream comprising carbon dioxide; repressurizing the fifth bed from the second pressure to a third pressure higher than the second pressure using a second portion of the first light product stream, a portion of the second light product stream, or a combination thereof; and repeating the cycle to generate the purified carbon dioxide stream comprising from about 80 vol.-% to about 95 vol.-% carbon dioxide.
Embodiment 14. The method of embodiment 13, or any of the preceding embodiments, wherein the light product stream comprises nitrogen.
Embodiment 15. The method of embodiment 13 or 14, or any of the preceding embodiments, wherein the third pressure is about the same as the first pressure.
Embodiment 16. The method of any of embodiments 1 to 3, or any of the preceding embodiments, wherein the purification process is temperature swing adsorption comprising at least three adsorbent beds wherein the temperature swing adsorption cycle comprises: passing the dilute carbon dioxide stream through the first bed at a first temperature to generate a light product stream; heating the second bed in a counter current mode from the first temperature to a second temperature higher than the first temperature to generate a heavy product stream comprising carbon dioxide; cooling the third bed from the second temperature to a third temperature lower than the second temperature; and repeating the cycle to generate the purified carbon dioxide stream comprising from about 80 vol.-% to about 95 vol.-% carbon dioxide.
Embodiment 17. The method of embodiment 16, or any of the preceding embodiments, wherein the light product stream comprises nitrogen.
Embodiment 18. The method of embodiment 16 or 17, or any of the preceding embodiments, wherein the third temperature is about the same as the first temperature.
Embodiment 19. The method of embodiment 1 or any of the preceding embodiments, wherein the purification process is temperature swing adsorption comprising at least five adsorbent beds wherein the temperature swing adsorption cycle comprises: passing the dilute carbon dioxide stream through the first bed at a first temperature to generate a light product stream; passing an effluent from the fourth bed through the second bed at a second temperature higher than the first temperature to generate a second light product stream; heating the third bed in a counter current mode from the second temperature to a third temperature higher than the second temperature to generate a heavy product stream comprising carbon dioxide; passing at least a portion of the first or a portion of the second light product stream through the fourth bed at the second temperature in a counter current mode generating the effluent stream from the fourth bed; cooling the fifth bed from the second temperature to a fourth temperature lower than the second temperature; and repeating the cycle to generate the purified carbon dioxide stream comprising from about 80 vol.-% to about 95 vol.-% carbon dioxide.
Embodiment 20. The method of embodiment 19, or any of the preceding embodiments, wherein the light product stream comprises nitrogen.
Embodiment 21. The method of embodiment 19 or 20, or any of the preceding embodiments, wherein the fourth temperature is about the same as the first temperature.
Embodiment 22. The method of embodiment 1, or any of the preceding embodiments, wherein the purification process is temperature swing adsorption comprising at least six adsorbent beds wherein the temperature swing adsorption cycle comprises: mixing the dilute carbon dioxide stream and an effluent from the third bed at a second temperature to preheat the dilute carbon dioxide stream to a first temperature and passing the mixture through the first bed at the first temperature to generate a first light product stream; passing an effluent from the fifth bed through the second bed in a counter current mode at the first temperature to generate a second light product stream; heating the third bed in a counter current mode from the first temperature to the second temperature higher than the first temperature to generate the effluent from the third bed at the second temperature; the fourth bed is desorbed at the second temperature to generate a heavy product stream comprising carbon dioxide; passing at least a portion of the first light product stream, a portion of the second light product stream, or both through the fifth bed at the first temperature in a co-current mode to generate the effluent stream from the fifth bed; cooling the sixth bed at the second temperature to a third temperature lower than the second temperature; and repeating the cycle is to generate the purified carbon dioxide stream comprising from about 80 vol.-% to about 95 vol.-% carbon dioxide.
Embodiment 23. The method of embodiment 22, or any of the preceding embodiments, wherein the light product stream comprises nitrogen.
Embodiment 24. The method of embodiment 1, or any of the preceding embodiments, wherein the purification process is a multi-stage membrane process comprising at least n membrane stages wherein a retentate of each membrane stage is passed to the feed of membrane stage n+1, and the permeate of each membrane stage is recycled to membrane stage n−1.
Embodiment 25. The method of embodiment 24, or any of the preceding embodiments, where n=2 or n=3.
Embodiment 26. The method of any of embodiments 1 to 25, wherein the purification process is a membrane suitable to generate the purified carbon dioxide stream.
Embodiment 27. The method of any of embodiments 1 to 26, wherein the dilute carbon dioxide stream is provided from an industrial process.
Embodiment 28. The method of any of embodiments 1 to 27, wherein the dilute carbon dioxide stream is a waste gas, flare gas, or flue gas.
Embodiment 29. The method of any of embodiments 1 to 28, wherein the microorganism is selected from a genus of Clostridium, Moorella, Carboxydothermus, Ruminococcus, Acetobacterium, Eubacterium, Butyribacterium, Oxobacter, Methanosarcina, Methanosarcina, Desulfotomaculum, and Cupriavidus.
Embodiment 30. The method of any of embodiments 1 to 29, wherein the target product is selected from an alcohol, an acid, a diacid, an alkene, a terpene, an isoprene, and alkyne.

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology.

All patents, patent applications, and published references cited herein are hereby incorporated by reference in their entirety. The claims are intended to cover the components and steps in any sequence which is effective to meet the objectives there intended unless the context specifically indicates the contrary.

The use of the terms “a” and “an” and “the” and similar terms are to be construed to cover both the singular and the plural, unless otherwise indicated herein or clearly contradicted by context. The terms “comprising,” “having,” “including,” and “containing” are to be construed as open-ended terms unless otherwise noted. The use of the alternative, such as the term “or”, should be understood to mean either one, both, or any combination thereof of the alternatives.

Recitation of ranges of values herein are merely intended to serve as a shorthand method of referring individually to each separate value falling within the range, unless otherwise indicated herein, and each separate value is incorporated into the specification as if it were individually recited herein. For example, any concentration range, percentage range, ratio range, integer range, size range, or thickness range is to be understood to include the value of any integer within the recited range and, when appropriate, fractions thereof (such as one tenth and one hundredth of an integer), unless otherwise indicated.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure, and does not pose a limitation on the scope of the disclosure unless otherwise claimed. No language in the specification should be construed as indicating any non-claimed element as essential to the practice of the disclosure.

Embodiments of this disclosure are described herein. Variations of those embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A method of carbon utilization comprising:
a. passing a dilute carbon dioxide stream to a purification zone wherein the dilute carbon dioxide stream comprises less than about 80 vol.-% carbon dioxide;
b. purifying the dilute carbon dioxide stream to generate a purified carbon dioxide stream comprising from about 80 vol.-% to about 95 vol.-% carbon dioxide, wherein the purifying is by a process selected from an amine absorption process, a temperature swing adsorption process, a pressure swing adsorption process, a membrane separation process, or any combination thereof;
c. passing hydrogen and the purified carbon dioxide stream to a gas fermentation zone and contacting with C1-fixing microorganism biocatalyst to ferment at least the hydrogen and the purified carbon dioxide stream and produce at least one target product; and
d. recovering the target product.

2. The method of claim 1 wherein the dilute carbon dioxide stream comprises less than about 90 vol.-% carbon dioxide, and the purified carbon dioxide stream comprises from about 90 vol.-% to about 95 vol.-% carbon dioxide.

3. The method of claim 1 wherein the C1-fixing microorganism is an acetogenic carboxydotrophic microorganism.

4. The method of claim 1 wherein the purification process is amine absorption and the solvent of the amine absorption process is selected from aqueous solutions of monoethanolamine, piperazine, diethanolamine, methyldiethanolamine, diglycolamine, 2-amino-2-methyl-1-propanol, or any combination thereof.

5. The method of claim 4 wherein the amine absorption purification process comprises an absorber column and a desorber column, wherein a rich solvent from the absorber column is preheated by heat exchange with regenerated solvent from the desorber column.

6. The method of claim 4 wherein the amine absorption purification process comprises an absorber column and a desorber column, and a rich solvent from the absorber column is split into two or more rich solvent streams and each rich solvent stream is passed to the desorber column.

7. The method of claim 1 wherein the purification process is pressure swing adsorption comprising at least four adsorbent beds wherein the pressure swing adsorption cycle comprises:
a. passing the dilute carbon dioxide stream through the first bed at a first pressure to generate a light product stream;
b. depressurizing the second bed in a counter current mode from the first pressure to a second pressure lower than the first pressure to generate a heavy product stream comprising carbon dioxide;
c. desorbing the third bed at the second pressure using a first portion of the light product stream to generate a second heavy product stream comprising carbon dioxide;
d. repressurizing the fourth bed from the second pressure to a third pressure higher than the second pressure using a second portion of the light product stream; and
e. repeating the cycle to generate the purified carbon dioxide stream comprising from about 80 vol.-% to about 95 vol.-% carbon dioxide.

8. The method of claim 7 wherein the third pressure is about the same as the first pressure.

9. The method of claim 1 wherein the purification process is pressure swing adsorption comprising at least four adsorbent beds wherein the pressure swing adsorption cycle comprises:
a. passing the dilute carbon dioxide stream through the first bed at a first pressure to generate a first light product stream;
b. passing a portion of a heavy product stream from the third bed through the second bed at the first pressure in a counter current mode to generate a second light product stream;
c. depressurizing the third bed in a counter current mode from the first pressure to a second pressure lower than the first pressure to generate the heavy product stream comprising carbon dioxide;
d. repressurizing the fourth bed from the second pressure to a third pressure higher than the second pressure using a portion of the first light product stream, the second light product stream, or a combination thereof; and
e. repeating the cycle to generate the purified carbon dioxide stream comprising from about 80 vol.-% to about 95 vol.-% carbon dioxide.

10. The method of claim 9 wherein the third pressure is about the same as the first pressure.

11. The method of claim 1 wherein the purification process is pressure swing adsorption comprising at least five adsorbent beds wherein the pressure swing adsorption cycle comprises:
a. passing the dilute carbon dioxide stream through the first bed at a first pressure to generate a first light product stream;
b. passing at least a portion of a first or a second heavy product stream through the second bed at the first pressure in a counter current mode to generate a second light product stream;
c. depressurizing the third bed in a counter current mode from the first pressure to a second pressure lower than the first pressure to generate the first heavy product stream comprising carbon dioxide;
d. desorbing the fourth bed at the second pressure using a first portion of the light product stream to generate a second heavy product stream comprising carbon dioxide;
e. repressurizing the fifth bed from the second pressure to a third pressure higher than the second pressure using a second portion of the first light product stream, a portion of the second light product stream, or a combination thereof; and
f. repeating the cycle to generate the purified carbon dioxide stream comprising from about 80 vol.-% to about 95 vol.-% carbon dioxide.

12. The method of claim 11 wherein the third pressure is about the same as the first pressure.

13. The method of claim 1 wherein the purification process is temperature swing adsorption comprising at least three adsorbent beds wherein the temperature swing adsorption cycle comprises:
a. passing the dilute carbon dioxide stream through the first bed at a first temperature to generate a light product stream;
b. heating the second bed in a counter current mode from the first temperature to a second temperature higher than the first temperature to generate a heavy product stream comprising carbon dioxide;
c. cooling the third bed from the second temperature to a third temperature lower than the second temperature; and
d. repeating the cycle is repeated to generate the purified carbon dioxide stream comprising from about 80 vol.-% to about 95 vol.-% carbon dioxide.

14. The method of claim 13 wherein the third temperature is about the same as the first temperature.

15. The method of claim 1 wherein the purification process is temperature swing adsorption comprising at least five adsorbent beds wherein the temperature swing adsorption cycle comprises:
a. passing the dilute carbon dioxide stream through the first bed at a first temperature to generate a light product stream;
b. passing an effluent from the fourth bed through the second bed at a second temperature higher than the first temperature to generate a second light product stream;
c. heating the third bed in a counter current mode from the second temperature to a third temperature higher than the second temperature to generate a heavy product stream comprising carbon dioxide;
d. passing at least a portion of the first or a portion of the second light product stream through the fourth bed at the second temperature in a counter current mode to generate the effluent stream from the fourth bed;
e. cooling the fifth bed from the second temperature to a fourth temperature lower than the second temperature; and
f. repeating the cycle to generate the purified carbon dioxide stream comprising from about 80 vol.-% to about 95 vol.-% carbon dioxide.

16. The method of claim 15 wherein the fourth temperature is about the same as the first temperature.

17. The method of claim 1 wherein the purification process is temperature swing adsorption comprising at least six adsorbent beds wherein the temperature swing adsorption cycle comprises:
a. Mixing the dilute carbon dioxide stream and an effluent from the third bed at a second temperature to preheat the dilute carbon dioxide stream to a first temperature and passing the mixture through the first bed at the first temperature to generate a first light product stream;
b. passing an effluent from the fifth bed through the second bed in a counter current mode at the first temperature to generate a second light product stream;
c. heating the third bed in a counter current mode from the first temperature to the second temperature higher than the first temperature to generate the effluent from the third bed at the second temperature;
d. desorbing the fourth bed at the second temperature to generate a heavy product stream comprising carbon dioxide;
e. passing at least a portion of the first light product stream, a portion of the second light product stream, or both through the fifth bed at the first temperature in a cocurrent mode to generate the effluent stream from the fifth bed;
f. cooling the sixth bed from the second temperature to a third temperature lower than the second temperature; and
g. repeating the cycle to generate the purified carbon dioxide stream comprising from about 80 vol.-% to about 95 vol.-% carbon dioxide.

18. The method of claim 1 wherein the purification process is a multi-stage membrane process comprising at least n membrane stages wherein a retentate of each membrane stage is passed to the feed of membrane stage n+1, and the permeate of each membrane stage is recycled to membrane stage n−1.

19. The method of claim 18 where n=2 or n=3.

20. The method of claim 1 wherein the purification process is a membrane suitable to generate the purified carbon dioxide stream.
